SEQUENCE LISTING (7 sequences)

SEQ ID NO: 1 - protein, 5 aa, Unknown
  Description of Unknown Enterokinase cleavage site sequence
DDDDK

SEQ ID NO: 2 - protein, 4 aa, Unknown
  Description of Unknown Factor Xa cleavage site sequence
IEGR

SEQ ID NO: 3 - protein, 6 aa, Unknown
  Description of Unknown Thrombin cleavage site sequence
LVPRGS

SEQ ID NO: 4 - protein, 8 aa, Unknown
  Description of Unknown Renin cleavage site sequence
HPFHLVIH

SEQ ID NO: 5 - protein, 4 aa, Unknown
  Description of Unknown Caspase protease cleavage site sequence
DEVD

SEQ ID NO: 6 - protein, 25 aa, Artificial Sequence
  Description of Artificial Sequence Synthetic peptide
TEYKLVVVGADGVGKSALTIQLIQN

SEQ ID NO: 7 - protein, 25 aa, Unknown
  Description of Unknown Wildtype KRAS G12D fragment sequence on 7
TEYKLVVVGAGGVGKSALTIQLIQN

(19) United States
(12) Patent Application Publication
(10) Pub. No.: US 2022/0389406 A1
(43) Pub. Date: Dec. 8, 2022

(54) COMPOSITIONS AND METHODS FOR T-CELL RECEPTOR IDENTIFICATION

(71) Applicant: RootPath Genomics, Inc., Watertown, MA (US)

(72) Inventors: Xi CHEN, Newton, MA (US); Ely PORTER, Medford, MA (US)

(21) Appl. No.: 17/747,195

(22) Filed: May 18, 2022

Related U.S. Application Data
(63) Continuation of application No. PCT/US2020/060998, filed on Nov. 18, 2020.
(60) Provisional application No. 62/937,595, filed on Nov. 19, 2019.

(51) Int. Cl. C12N 15/10; G01N 33/50

(52) U.S. Cl. CPC C12N 15/1037; G01N 33/505; G01N 2333/7051

(57) ABSTRACT

The present disclosure provides compositions and methods for identifying target-reactive T-cell receptors (TCRs). The identification may be based on comparing the frequency of a TCR before and after a marker-based selection. The identification may also be based on comparing the frequency of a TCR in a pool of cells stimulated by mutant and wildtype antigens.

### CROSS-REFERENCE

This application is a continuation of International Application No. PCT/US2020/060998, filed on Nov. 18, 2020, which claims the benefit of U.S. Provisional Patent Application No. 62/937,595, filed on Nov. 19, 2019, the entire contents of each of which is entirely incorporated herein by reference.

### SEQUENCE LISTING

The instant application contains a Sequence Listing which has been submitted electronically in ASCII format and is hereby incorporated by reference in its entirety. Said ASCII copy, created on May 17, 2022, is named 53563_707_301_SL.txt and is 2,002 bytes in size.

### BACKGROUND OF THE INVENTION

The T-cell receptor (TCR) is responsible for the recognition of the antigen-major histocompatibility complex, leading to the initiation of an inflammatory response. Many T cell subsets exist, including cytotoxic T cells and helper T cells. Cytotoxic T cells (also known as CD8+ T cells) kill abnormal cells, for example virus-infected or tumor cells. Helper T cells (also known as CD4+ T cells) aid in the activation and maturation of other immune cells. Both cytotoxic and helper T cells carry out their function subsequent to the recognition of specific target antigens which triggers their respective responses. The antigen specificity of a T cell can be defined by the TCR expressed on the surface of the T cell. T cell receptors are heterodimer proteins composed of two polypeptide chains, most commonly an alpha and beta chain, but a minority of T cells can express a gamma and delta chain. The specific amino acid sequence of the TCR and the resultant three-dimensional structure defines the TCR antigen specificity and affinity. The amino acid and coding DNA sequences of the TCR chains for any individual T cell are almost always unique or at very low abundance in an organism's entire TCR repertoire, since there are a vast number of possible TCR sequences. This large sequence diversity is achieved during T cell development through a number of cellular mechanisms and may be a critical aspect of the immune system's ability to respond to a huge variety of potential antigens.

Analyzing the TCR repertoire may help to gain a better understanding of the immune system features and of the etiology and progression of diseases, in particular those with unknown antigenic triggers.

### SUMMARY OF THE INVENTION

Recognized herein is a need to develop methods to identify TCR clones from sequencing data in a high-throughput manner.

In an aspect, the present disclosure provides a method for identifying a T-cell receptor (TCR), comprising: (a) providing a plurality of T cells expressing a plurality of TCRs, wherein each T cell of the plurality of T cells expresses a cognate pair of a TCR of the plurality of TCRs; (b) pairing a first polynucleotide encoding a first TCR chain and a second polynucleotide encoding a second TCR chain of the cognate pair of the TCR of each T cell, thereby generating a plurality of polynucleotide pairs; (c) delivering polynucleotides comprising the plurality of polynucleotide pairs into a plurality of recipient cells, wherein each recipient cell comprises a polynucleotide comprising at least one polynucleotide pair of the plurality of polynucleotide pairs; (d) expressing the plurality of polynucleotide pairs in the plurality of recipient cells; (e) sequencing a TCR repertoire of the plurality of recipient cells and determining a frequency of a TCR of the TCR repertoire in the plurality of recipient cells; (f) contacting the plurality of recipient cells with one or more antigens, thereby activating a marker in a subset of the plurality of recipient cells; (g) isolating the subset of the plurality of recipient cells based on the marker; (h) sequencing a TCR repertoire of the subset of the plurality of recipient cells and determining a frequency of a TCR of the TCR repertoire in the subset of the plurality of recipient cells; and (i) identifying a TCR having a frequency in the subset of the plurality of recipient cells that is higher than its frequency in the plurality of recipient cells.

In some embodiments, the frequency of the TCR in the subset of the plurality of recipient cells is at least 1.5-fold, 1.8-fold, 2.0-fold, 2.5-fold, 3.0-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10-fold, 15-fold, 20-fold, 30-fold, 40-fold, 50-fold, 60-fold, 70-fold, 80-fold, 90-fold, 100-fold, or more higher than its frequency in the plurality of recipient cells. In some embodiments, the marker is a T-cell activation marker. In some embodiments, the marker is a reporter protein. In some embodiments, the reporter protein is a fluorescent protein. In some embodiments, the marker is a cell surface protein, an intracellular protein or a secreted protein. In some embodiments, the marker is the intracellular protein or the secreted protein, and wherein the method further comprises, prior to isolating, fixing and/or permeabilizing the plurality of recipient cells. In some embodiments, the method further comprises contacting the plurality of recipient cells with a Golgi blocker. In some embodiments, the secreted protein is a cytokine. In some embodiments, the cytokine is IFN-γ, TNF-alpha, IL-17A, IL-2, IL-3, IL-4, GM-CSF, IL-10, IL-13, granzyme B, perforin, or a combination thereof. In some embodiments, the cell surface protein is CD39, CD69, CD103, CD25, PD-1, TIM-3, OX-40, 4-1BB, CD137, CD3, CD28, CD4, CD8, CD45RA, CD45RO, GITR, FoxP3, or a combination thereof. In some embodiments, the one or more antigens are represented on one or more antigen presenting cells (APCs), MHC tetramers, nanoparticles or any combination thereof. In some embodiments, the one or more APCs are or derived from one or more cells isolated from a subject. In some embodiments, the one or more APCs are one or more artificial APCs (aAPCs). In some embodiments, the one or more APCs express MHC molecules exogenous to the one or more APCs. In some embodiments, the one or more APCs are one or more cancer cells, a tumorsphere, a tumoroid, or a derivative thereof. In some embodiments, the one or more APCs comprise an antigen coding DNA or RNA. In some embodiments, the RNA is an mRNA vector. In some embodiments, the mRNA vector is a self-amplifying mRNA. In some embodiments, the one or more APCs are pulsed with the one or more antigens. In some embodiments, the one or more antigens are derived from one or more tumor antigens. In some embodiments, the one or more tumor antigens are one or more tumor-associated antigens (TAAs) or tumor-specific antigens (TSAs). In some embodiments, the one or more antigens comprise NY-ESO-1, WT-1, SSX, MAGE-A3, PRAME, Survivin, Gp100, Melan-A/Mart-1, tyrosinase, PSA, CEA, mammaglobin, p53, HER2/neu, hTERT, proteinase-3, mucin-1, or MAGE-A4. In some embodiments, the one or more antigens comprises a genetic abnormality or an epigenetic abnormality. In some embodiments, the genetic abnormality comprises a point mutation, a fusion, a deletion, an insertion, a frameshift, an intron inclusion or an alternative splicing. In some embodiments, the one or more antigens are upregulated (e.g., the expression level of the antigens are upregulated) in a cancer cell compared to a healthy cell. In some embodiments, the method further comprises selecting the TCR identified in (i) from the TCR repertoire of the plurality of recipient cells or the TCR repertoire of the subset of the plurality of recipient cells. In some embodiments, selecting the TCR identified in (i) comprises amplifying the TCR. In some embodiments, the polynucleotide in (c) comprises a barcode.

In another aspect, the present disclosure provides a method for identifying a T-cell receptor (TCR), comprising: (a) providing a plurality of T cells expressing a plurality of TCRs, wherein each T cell of the plurality of T cells expresses a cognate pair of a TCR of the plurality of TCRs; (b) pairing a first polynucleotide encoding a first TCR chain and a second polynucleotide encoding a second TCR chain of the cognate pair of the TCR of each T cell, thereby generating a plurality of polynucleotide pairs; (c) delivering polynucleotides comprising the plurality of polynucleotide pairs into a first plurality of recipient cells and a second plurality of recipient cells, where each recipient cell of the first or the second plurality of recipient cells comprises a polynucleotide comprising at least one polynucleotide pair of the plurality of polynucleotide pairs; (d) expressing the plurality of polynucleotide pairs in the first and second plurality of recipient cells; (e) contacting the first plurality of recipient cells with a first antigen, thereby activating a first marker of a first subset of the first plurality of recipient cells, and contacting the second plurality of recipient cells with a second antigen, thereby activating a second marker of a second subset of the second plurality of recipient cells; (f) isolating the first subset based on the first marker and the second subset based on the second marker; (g) sequencing a TCR repertoire of the first subset of the first plurality of recipient cells and a TCR repertoire of the second subset of the second plurality of recipient cells, and determining a frequency of a TCR of the TCR repertoire in the first subset of the first plurality of recipient cells and a frequency of a TCR of the TCR repertoire in the second subset of the second plurality of recipient cells; and (h) identifying a TCR having a frequency in the first subset of the first plurality of recipient cells that is higher than its frequency in the second subset of the second plurality of recipient cells.

In some embodiments, the frequency of the TCR in the first subset of the first plurality of recipient cells is at least 1.5-fold, 1.8-fold, 2.0-fold, 2.5-fold, 3.0-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10-fold, 15-fold, 20-fold, 30-fold, 40-fold, 50-fold, 60-fold, 70-fold, 80-fold, 90-fold, 100-fold, or more higher than its frequency in the second subset of the second plurality of recipient cells. In some embodiments, the first marker and the second marker are the same. In some embodiments, the first marker and the second marker are different. In some embodiments, the second antigen is not homologous to the first antigen. In some embodiments, the first antigen and the second antigen are derived from the same protein. In some embodiments, the first antigen comprises a mutated sequence and the second antigen comprises a wildtype sequence. In some embodiments, the first antigen is derived from a cancer cell and the second antigen is derived from a healthy cell. In some embodiments, the method further comprises, prior to contacting, sequencing a TCR repertoire of the plurality of recipient cells, and determining a frequency of a TCR of the TCR repertoire in the plurality of recipient cells. In some embodiments, the method further comprises selecting the TCR identified in (h) from a TCR repertoire of the first plurality of recipient cells or the TCR repertoire of the first subset of the first plurality of recipient cells. In some embodiments, selecting the TCR identified in (h) comprises amplifying the TCR. In some embodiments, the polynucleotide in (c) comprises a barcode.

In some embodiments, the plurality of T cells are isolated from a subject. In some embodiments, the plurality of T cells are tumor-infiltrating lymphocytes.

In another aspect, the present disclosure provides a method for identifying a T-cell receptor (TCR), comprising: (a) providing a plurality of cells expressing a plurality of TCRs, each cell of the plurality of cells expressing a TCR of the plurality of TCRs, wherein the plurality of TCRs comprises at least 5 different cognate pairs and comprises V regions from a plurality of V genes, and wherein the plurality of TCRs are exogenous to the plurality of cells; (b) sequencing a TCR repertoire of the plurality of cells, and determining a frequency of a TCR of the TCR repertoire in the plurality of cells; (c) contacting the plurality of cells with one or more antigens, thereby activating a marker of a subset of the plurality of cells; (d) isolating the subset of the plurality of cells based on the marker; (e) sequencing a TCR repertoire of the subset of the plurality of cells, and determining a frequency of a TCR of the TCR repertoire in the subset of the plurality of cells; and (f) identifying a TCR having a frequency in the subset of the plurality of cells that is higher than its frequency in the plurality of cells. In some embodiments, the method further comprising selecting the TCR identified in (f) from the TCR repertoire of the plurality of cells or the TCR repertoire of the subset of the plurality of cells. In some embodiments, selecting the TCR identified in (f) comprises amplifying the TCR. In some embodiments, a sequence encoding a TCR of the plurality of TCRs in (a) comprises a barcode.

In another aspect, the present disclosure provides a method for identifying a T-cell receptor (TCR), comprising: (a) providing a first plurality of cells expressing a plurality of TCRs and a second plurality of cells expressing the plurality of TCRs, each cell of the first or the second plurality of cells expressing a TCR of the plurality of TCRs, wherein the plurality of TCRs comprises at least 5 different cognate pairs and comprises V regions from a plurality of V genes, and wherein the plurality of TCRs are exogenous to the first or the second plurality of cells; (b) contacting the first plurality of cells with a first antigen, thereby activating a first marker of a first subset of the first plurality of cells, and contacting the second plurality of cells with a second antigen, thereby activating a second marker of a second subset of the second plurality of cells; (c) isolating the first subset based on the first marker and the second subset based on the second marker; (d) sequencing a TCR repertoire of the first subset of the first plurality of cells and a TCR repertoire of the second subset of the second plurality of cells, and determining a frequency of a TCR of the TCR repertoire in the first subset of the first plurality of cells and a frequency of a TCR of the TCR repertoire in the second subset of the second plurality of cells; and (e) identifying a TCR having a frequency in the first subset of the first plurality of cells that is higher than its frequency in the second subset of the second plurality of cells. In some embodiments, the method further comprising selecting the TCR identified in (e) from a TCR repertoire of the first plurality of cells or the TCR repertoire of the first subset of the first plurality of cells. In some embodiments, selecting the TCR identified in (e) comprises amplifying the TCR. In some embodiments, a sequence encoding a TCR of the plurality of TCRs in (a) comprises a barcode.

In some embodiments, the frequency is determined by sequencing reads of a TCR divided by total sequencing reads of the TCR repertoire. In some embodiments, the identified TCR is a target-reactive TCR.

In another aspect, the present disclosure provides a pharmaceutical composition comprising a TCR or a cell expressing a TCR identified by any one of the methods described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure”, “Fig.”, and “FIGURE” herein) of which:

FIG. 1 depicts an example workflow of T-cell receptor identification described herein.

FIG. 2 depicts an example workflow of T-cell receptor identification by comparing frequencies of a TCR in a pre-selection pool and a post-selection pool.

FIG. 3 depicts an example workflow of T cell receptor identification by comparing frequencies of a TCR in a first post-selection pool and a second post-selection pool.

FIG. 4 depicts experimental data for T-cell receptor identification using the methods described herein. The data show the frequency of each TCR in the post-real-selection polyclonal TCR-T cells (Y-axis) and pre-selection polyclonal TCR-T cells (X-axis).

FIG. 5 depicts experimental data for T-cell receptor identification using the methods described herein. The data show the frequency of each TCR in the post-mock-selection polyclonal TCR-T cells (Y-axis) and pre-selection polyclonal TCR-T cells (X-axis).

FIG. 6 depicts experimental data for T-cell receptor identification using the methods described herein. The data show the enrichment factor of TCR in the ‘real’ co-culture (Y-axis) and the ‘mock’ co-culture (X-axis). The size of the dot shows the frequency of the TCR in the pre-selection polyclonal TCR-T cells.

### DETAILED DESCRIPTION OF THE INVENTION

In this disclosure, the use of the singular includes the plural unless specifically stated otherwise. Also, the use of “or” means “and/or” unless stated otherwise. Similarly, “comprise,” “comprises,” “comprising” “include,” “includes,” and “including” are not intended to be limiting.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “immunoreceptor” refers to a receptor protein or a receptor protein complex that an immune cell produces to recognize its target. The target may be an antigen or a portion thereof (e.g., an epitope). The antigen can be a protein or a peptide. The target may be an MI-1C-bound peptide. Examples of immunoreceptors include B cell receptors (BCRs), antibodies (used interchangeably with “immunoglobulins”), and T cell receptors (TCRs).

The term “immunoreceptor chain” refers to a polypeptide that functions as a subunit of an immunoreceptor. Examples of immunoreceptor chains include the heavy chain of an immunoglobulin (Ig), the light chain of an immunoglobulin, the alpha chain of a TCR, the beta chain of a TCR, the gamma chain of a TCR, the delta chain of a TCR.

The term “bipartite immunoreceptor” refers to an immunoreceptor that is formed by polypeptides encoded by two genes. In cells, the two genes may be located on different loci of a chromosome, or different chromosomes. The two genes can be rearranged genes, such as V(D)J-rearranged genes. V(D)J-rearranged genes can be generated through a mechanism called V(D)J recombination, which occurs in the primary lymphoid organs and in a nearly random fashion rearranges variable (V), joining (J), and in some cases, diversity (D) gene segments. Examples of bipartite immunoreceptor include, but are not limited to, BCR (encoded by rearranged heavy chain gene and rearranged light chain gene), antibody (encoded by rearranged heavy chain gene and rearranged light chain gene), and TCR (encoded by rearranged TRA gene and rearranged TRB gene, or encoded by rearranged TRG gene and rearranged TRD gene).

The term “source TCR-expressing cell” refers to TCR-expressing cells (e.g., T cells) whose TCRs can be cloned into vectors for expression.

The term “recipient cell” refers to a cell to which an immunoreceptor-expressing vector (e.g., TCR-expressing vector) can be functionally introduced. The phrase “functionally introduced” means that the immunoreceptor encoded in the immunoreceptor-expressing vector can be expressed in the recipient cell. Examples of recipient cells include, but are not limited to, CD45+ cells, T cells, B cells, macrophages, natural killer (NK) cells, stem cells, bacterial cells, yeast cells, and cell lines.

The terms “enriching,” “isolating,” “separating,” “sorting,” “purifying,” “selecting” or equivalents thereof can be used interchangeably and refer to obtaining a subsample with a given property from a sample. For example, enriching can comprise obtaining a cell population or cell sample that contains at least about 2%, 5%, 10%, 20%, 30%, 40%, 50%, 55%, 60%, 65%, 70%, 75%, 80%, 85%, 90%, 95%, or 100% of the desired cell lineage or a desired cell having a certain cell phenotype, e.g., expressing a certain cell marker or not expressing a certain cell marker gene characteristic of that cell phenotype.

The term “TCR repertoire,” as used herein, refers to a collection of or a library of TCRs. In some cases, the TCR repertoire of a sample or a plurality of cells comprises all or substantially all TCRs within the sample or expressed by the plurality of cells. In some cases, the TCR repertoire of a sample or a plurality of cells may comprise at least about 80%, 85%, 90%, 95%, or 100% of the TCRs within the sample or expressed by the plurality of cells. In some cases, the TCR repertoire of a sample or a plurality of cells may comprise at least about 80%, 85%, 90%, 95%, or 100% of unique TCRs within the sample or expressed by the plurality of cells. In various aspects, analyzing a TCR repertoire can comprise sequencing the sequences encoding the TCRs of the TCR repertoire.

### Overview

A polyclonal population of source TCR-expressing cells can be converted into a polyclonal population of TCR-programmed recipient cells, where the engineered TCR repertoire of the TCR-programmed recipient cells can comprise the natural TCR repertoire (e.g., cognate pair combinations of the TCR chains) of the source TCR-expressing cells. The bipartite nature of TCR made this task difficult with conventional technologies. The methods provided herein can be used to overcome these difficulties. There can be several advantages of using the TCR-programmed recipient cells over using the source TCR-expressing cells. For example, the TCR-programmed recipient cells may be prepared at larger numbers, may have a more ideal functional characteristic, may be in a more ideal epigenetic state, may have a more uniform genetic or phenotypic background, may be engineered to express additional agents to enhance antigen-dependent stimulation, or may be engineered to express reporter genes to aid selection. The TCR-programmed recipient cells can be used to identify putative antigen-reactive TCRs. The methods for identifying putative antigen-reactive TCRs can comprise determining a frequency of a particular TCR before and/or after selection. The methods provided herein can overcome the limitations (e.g., false positives) associated with conventional selection methods based on cell markers. For example, cell sorting using fluorescence-activated cell sorting (FACS) or magnetic activated cell sorting (MACS) may result in false positives because many selected cells may express the cell markers but may not be reactive to the target antigens. Using the methods provided herein can greatly reduce the false positive rate and increase the change of identify true antigen-reactive cells.

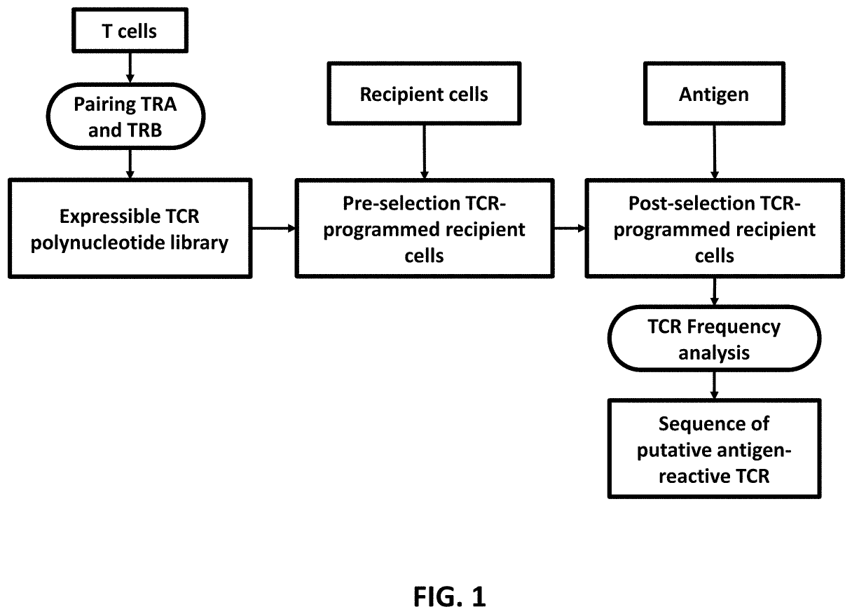

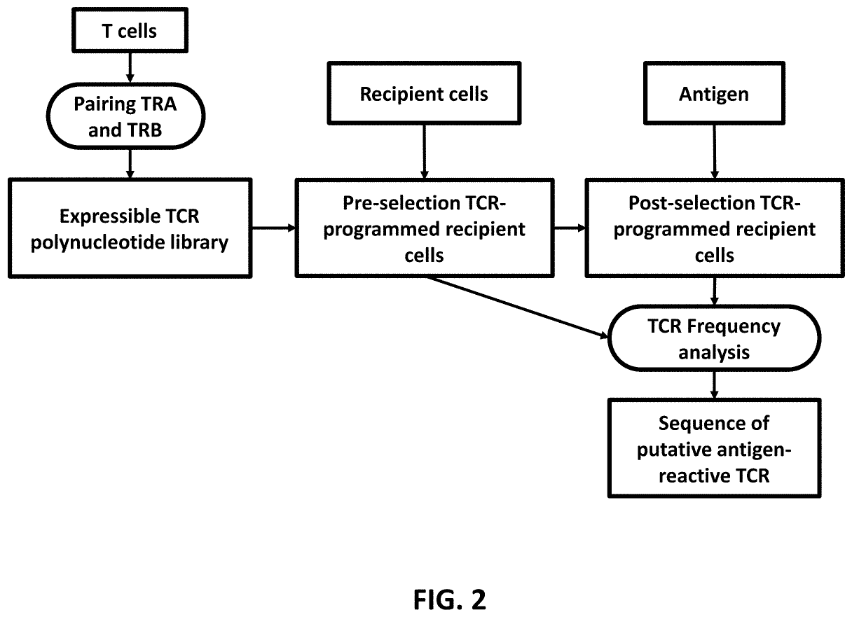

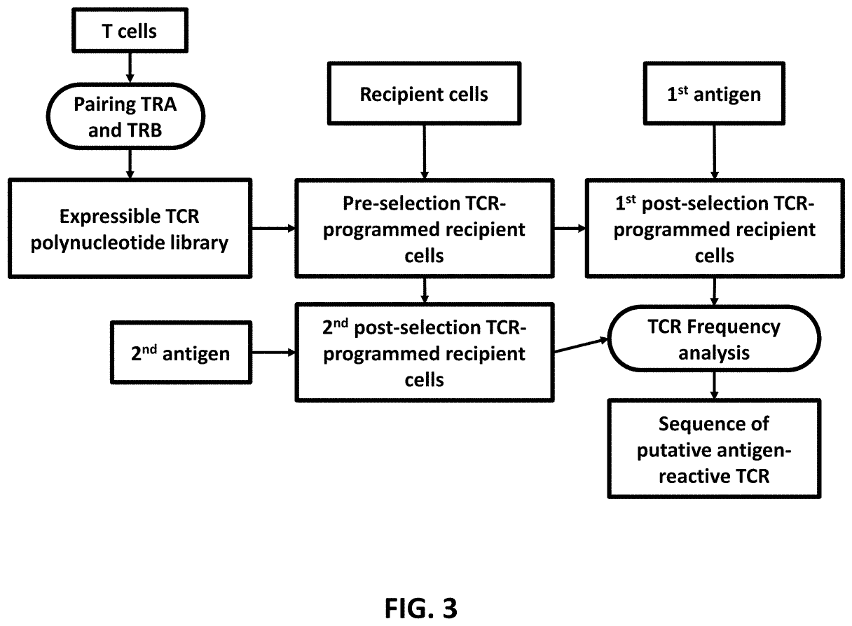

As an example of the methods provided herein, the TCR-programmed recipient cells can be contacted with one or more antigens (e.g., one or more antigens in complexed with MHCs). Subsequent to contacting with the one or more antigens, the TCR-programmed recipient cells can be subject to selection (e.g., using fluorescence-activated cell sorting (FACS), magnetic activated cell sorting (MACS), panning or other method) to obtain post-selection TCR-programmed recipient cells. The post-selection TCR-programmed recipient cells can be sequenced. The sequencing reads can be used to measure the frequency or relative abundance of each TCR (see “TCR frequency analysis” in FIGS. 1-3). The frequency of a TCR can be defined as the number of original molecules (e.g., cDNAs) encoding a TCR (e.g., a TCR with a unique sequence) divided by the number of all original molecules observed in one sample. The TCR frequency analysis can be used to identify putative target-reactive TCRs (e.g., antigen-reactive TCRs). In some cases, prior to selection, the TCR-programmed recipient cells (e.g., pre-selection TCR-programmed recipient cells) may be sequenced and subject to TCR frequency analysis. The frequency of a TCR in the pre-selection pool and the post-selection pool can be used to identify the target-reactive TCRs (FIG. 2). In some cases, the pre-selection TCR-programmed recipient cells can be contacted with a first antigen and a second antigen, where the second antigen may be a variant of the first antigen. The second antigen may contain one or more mutations compared with the first antigen. Subsequent to contacting with the first and second antigens, the TCR-programmed recipient cells can be subject to selection to obtain a first and a second pool of post-selection TCR-programmed recipient cells. The first and the second pool of post-selection TCR-programmed recipient cells can be sequenced and subject to TCR frequency analysis. The frequency of a TCR in the first pool and the second pool can be used to identify the target-reactive TCRs (FIG. 3). The TCR-programmed recipient cells and the methods to identify putative antigen- or tumor-reactive TCRs may be used in various applications.

The methods provided herein can be used to identify a plurality of antigen- or tumor-reactive TCRs from a population of TCRs. A method provided herein can be used to identify a plurality of tumor-reactive T-cell receptors (TCRs) from a population of TCRs, wherein the population of TCRs comprise at least about 20, 30, 50, 100, 1,000, 10,000, 100,000, 1,000,000, 10,000,000, or more different cognate pairs. A plurality of cells expressing the plurality of cognate TCRs or a subset thereof can be used to identify one or a plurality of tumor-reactive TCRs. The plurality of cognate TCRs or a subset thereof may be exogenous to the plurality of cells. The plurality of TCRs or a subset thereof can comprise at least 5, at least 10, at least 15, or at least 20 different cognate pairs. In some cases, the plurality of tumor-reactive TCRs or a subset thereof may comprise greater than or equal to about 5, 10, 20, 30, 40, 50, 60, 100, 200, 300, 400, 500, 1,000, 1,500, 2,000, 2,500, 3,000, 3,500, or 4,000, or more different cognate pairs. Each TCR of the plurality of tumor-reactive TCRs may be specific to a different epitope or a different protein or may comprise a different (i) TCR alpha CDR3 sequence, (ii) TCR beta CDR3 variable domain sequence, (iii) TCR alpha variable domain sequence, (iv) TCR beta variable domain sequence, or (v) TCR alpha and TCR beta variable domain sequence in combination. The method can further comprise isolating a population of T cells expressing the population of TCRs from the subject. The different cognate pairs of TCRs may comprise V regions from at least 5, 10, 15, 20, or more different V genes.

The methods provided herein can identify antigen- or tumor-reactive TCRs from a sample with a small sample size, such as having at most about 100,000, 10,000, 1,000, 100, or less cells. A method of identifying antigen- or tumor-reactive TCRs provided herein can comprise isolating a population of T cells from the subject that express a population of T-cell receptors (TCRs), wherein the population of T cells comprises at most about 1,000 cells, 10,000 cells, or 100,000 cells.

### Expression Vectors

The TCRs from source TCR-expressing cells can be used to generate an expressible TCR polynucleotide library and delivered into recipient cells for expression. The expressible TCR polynucleotide library can comprise a library of vectors. A polynucleotide of the expressible TCR polynucleotide library may be delivered into a recipient cell as a linear or circular nucleic acid molecule. In some cases, the polynucleotide can be delivered into a recipient cell by electroporation. In some cases, the polynucleotide can be delivered into a recipient cell by a carrier such as a cationic polymer.

A polynucleotide molecule encoding a TCR can be delivered into a recipient cell described herein. The polynucleotide molecule can be deoxyribonucleic acid (DNA), ribonucleic acid (RNA), or a combination thereof. The polynucleotide molecule can be mRNA. The polynucleotide molecule may comprise an analog of a nucleic acid.

The polynucleotide molecule encoding the TCR can comprise a full coding sequence of a TCR or a portion thereof. The polynucleotide molecule encoding the TCR can comprise a coding sequence of a cognate pair of a TCR including a TCR alpha chain or a TCR beta chain, or a TCR gamma chain or a TCR delta chain. The polynucleotide molecule encoding a TCR can be at least about 200, 300, 400, 500, 600, 700, 800, 900, 1,000, 2,000, 3,000, 4,000, 5,000, 6,000, 7,000, 8,000, 9,000, 10,000 or more nucleotides in length.

The polynucleotide molecule encoding a TCR can be delivered into the recipient cell by a vector. The vector may comprise a marker gene (e.g., GFP) for the purpose of determining vector titers and transfection or transduction efficiency. In some cases, the cells can be transfected or transduced at a low multiplicity of infection (MOI) such as at MOI of at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 or more. In some cases, the cells can be transfected or transduced at MOI of at most about 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5 or less. In some cases, a library of polynucleotide molecules encoding a plurality of different TCRs can be delivered into a plurality of recipient cells such that an individual recipient cell can express only one, two, three, four, five, six, seven, eight or more TCRs. In some cases, a library of polynucleotide molecules encoding a plurality of different TCRs can be delivered into a plurality of recipient cells such that an individual recipient cell can express at most about eight, seven, six, five, four, three, two or one TCR(s). In some cases, an individual recipient cell can express only one TCR (e.g., a unique TCR pair). For example, a lentiviral vector or self-amplifying RNA may be used to deliver one copy of the polynucleotide molecule encoding a unique TCR into one recipient cell. For another example, the polynucleotide molecule encoding a unique TCR can be genetically knocked in into the genome of the recipient cell by any available gene editing technology, e.g., CRISPR.

The TCR can be expressed from a vector such as plasmid, transposon (e.g., Sleeping Beauty, Piggy Bac), and a viral vector (e.g., adenoviral vector, AAV vector, retroviral vector and lentiviral vector). Additional examples of a vector include a shuttle vector, a phagemide, a cosmid and an expression vector. Non-limiting examples of plasmid vectors include pUC, pBR322, pET, pBluescript, and variants thereof. Further, a vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. In some cases, a vector is a nucleic acid molecule as introduced into a recipient cell, thereby producing a transformed recipient cell. A vector may include nucleic acid sequences that permit it to replicate in a recipient cell, such as an origin of replication. A vector may also include one or more selectable marker genes and other genetic elements. A vector can be an expression vector that includes a paired TCR-encoding polynucleotide according to the present disclosure operably linked to sequences allowing for the expression of the TCR. A vector can be a viral or a non-viral vector, such a retroviral vector (including lentiviral vectors), adenoviral vectors including replication competent, replication deficient and gutless forms thereof, adeno-associated virus (AAV) vectors, simian virus 40 (SV-40) vectors, bovine papilloma vectors, Epstein-Barr vectors, herpes vectors, vaccinia vectors, Moloney murine leukemia vectors, Harvey murine sarcoma virus vectors, murine mammary tumor virus vectors, Rous sarcoma virus vectors and nonviral plasmids. Baculovirus vectors can be suitable for expression in insect cells.

In some embodiments, the vector is a self-amplifying RNA replicon, also referred to as self-replicating (m)RNA, self-replication (m)RNA, self-amplifying (m)RNA, or RNA replicon. The self-amplifying RNA replicon is an RNA that can replicate itself. In some embodiments, the self-amplifying RNA replicon can replicate itself inside of a cell. In some embodiments, the self-amplifying RNA replicon encodes an RNA polymerase and a molecule of interest. The RNA polymerase may be a RNA-dependent RNA polymerase (RDRP or RdRp). The self-amplifying RNA replicon may also encode a protease or an RNA capping enzyme. In some embodiments, the self-amplifying RNA replicon vector is of or derived from the Togaviridae family of viruses known as alphaviruses which can include Eastern Equine Encephalitis virus (EEE), Venezuelan Equine Encephalitis virus (VEE), Everglades virus, Mucambo virus, Pixuna virus, Western Equine Encephalitis virus (WEE), Sindbis virus, South African Arbovirus No. 86, Semliki Forest virus, Middelburg virus, Chikungunya virus, Onyong-nyong virus, Ross River virus, Barmah Forest Virus, Getah Virus, Sagiyama virus, Bebaru virus, Mayaro virus, Una virus, Aura virus, Whataroa virus, Babanki virus, Kyzylagach virus, Highlands J Virus, Fort Morgan virus, Ndumu virus, Buggy Creek virus, and any other virus classified by the International Committee on Taxonomy of Viruses (ICTV) as an alphavirus. In some embodiments, the self-amplifying RNA replicon is or contains parts from an attenuated form of the alphavirus, such as the VEE TC-83 vaccine strain. In some embodiments, the self-amplifying RNA replicon vector is an attenuated form of the virus that allows for expression of the molecules of interests without cytopathic or apoptotic effects to the cell. In some embodiments, the self-amplifying RNA replicon vector has been engineered or selected in vitro, in vivo, ex vivo, or in silica for a specific function (e.g., prolonged or increased TCR expression) in the host cell, target cell, or organism. For example, a population of host cells harboring different variants of the self-amplifying RNA replicon can be selected based on the expression level of one or more molecules of interested (encoded in the self-amplifying RNA replicon or in the host genome) at different time point. In some embodiments, the selected or engineered self-amplifying RNA replicon has been modified to reduce the type I interferon response, the innate antiviral response, or the adaptive immune response from the host cell or organism which results in the RNA replicon's protein expression persisting longer or expressing at higher levels in the host cell, target cell, or organism. In some embodiments, this optimized self-amplifying RNA replicon sequence is obtained from an individual cell or population of cells with the desired phenotypic trait (e.g., higher or more sustained expression of the molecules of interest, or reduced innate antiviral immune response against the vector compared to the wildtype strains or the vaccine strains). In some embodiments, the cells harboring the desired or selected self-amplifying RNA replicon sequence are obtained from a subject (e.g., a human or an animal) with beneficial response characteristics (e.g., an elite responder or subject in complete remission) after being treated with a therapeutic agent comprising a self-amplifying RNA replicon. In some embodiments, the self-amplifying RNA replicon vector can express additional agents. In some embodiments, the additional agents include cytokines such as IL-2, IL-12, IL-15, IL-10, GM-CSF, TNF alpha, granzyme B, or a combination thereof. In some embodiments, the additional agent is capable of modulating the expression of the TCR, either by directly affecting the expression of the TCR or by modulating the host cell phenotype (e.g., inducing apoptosis or expansion). In some embodiments, the self-amplifying RNA replicon can contain one or more sub-genomic sequence(s) to produce one or more sub-genomic polynucleotide(s). In some embodiments, the sub-genomic polynucleotides act as functional mRNA molecules for translation by the cellular translation machinery. A sub-genomic polynucleotide can be produced via the function of a defined sequence element (e.g., a sub-genomic promoter or SGP) on the self-amplifying RNA replicon that directs a polymerase to produce the sub-genomic polynucleotide from a sub-genomic sequence. In some embodiments, the SGP is recognized by an RNA-dependent RNA polymerase (RDRP or RdRp). In some embodiments, multiple SGP sequences are present on a single self-amplifying RNA replicon and can be located upstream of sub-genomic sequence encoding for a TCR, a constituent of the TCR, or an additional agent. In some embodiments, the nucleotide length or composition of the SGP sequence can be modified to alter the expression characteristics of the sub-genomic polynucleotide. In some embodiments, non-identical SGP sequences are located on the self-amplifying RNA replicon such that the ratios of the corresponding sub-genomic polynucleotides are different from instances where the SGP sequences are identical. In some embodiments, non-identical SGP sequences direct the production of a TCR and an additional agent (e.g., a cytokine) such that they are produced at a ratio relative to one another that leads to increased expression of the TCR, increased or faster expansion of the target cell without cytotoxic effects to the target cell or host, or dampens the innate or adaptive immune response against the RNA replicon. In some embodiments, the location of the sub-genomic sequences and SGP sequences relative to one another and the genomic sequence itself can be used to alter the ratio of sub-genomic polynucleotides relative to one another. In some embodiments, the SGP and sub-genomic sequence encoding the TCR can be located downstream of an SGP and sub-genomic region encoding the additional agent such that the expression of the TCR is substantially increased relative to the additional agent. In some embodiments, the RNA replicon or SGP has been selected or engineered to express an optimal amount of the cytokine such that the cytokine promotes the expansion of the T cell or augments the therapeutic effect of the TCR but does not cause severe side effects such as cytokine release syndrome, cytokine storm, or neurological toxicity.

In some embodiments, provided herein is a vector comprising a paired TCR-encoding polynucleotide encoding a TCRα chain and a TCRβ chain. In some embodiments, provided herein is a vector comprising a paired TCR-encoding polynucleotide encoding a TCRγ chain and a TCR chain. In some embodiments, the vector is a self-amplifying RNA replicon, plasmid, phage, transposon, cosmid, virus, or virion. In some embodiments, the vector is a viral vector. In some embodiments, the vector is derived from a retrovirus, lentivirus, adenovirus, adeno-associated virus, herpes virus, pox virus, alpha virus, vaccina virus, hepatitis B virus, human papillomavirus or a pseudotype thereof. In some embodiments, the vector is a non-viral vector. In some embodiments, the non-viral vector can be formulated into a nanoparticle, a cationic lipid, a cationic polymer, a metallic nanopolymer, a nanorod, a liposome, a micelle, a microbubble, a cell-penetrating peptide, or a liposphere.

The expression of the two TCR chains can be driven by two promoters or by one promoter. In some cases, two promoters are used. In some cases, the two promoters, along with their respective protein-coding sequences for the two chains, can be arranged in a head-to-head, a head-to-tail, or a tail-to-tail orientation. In some cases, one promoter is used. The two protein-coding sequences can be linked, optionally in frame, such that one promoter can be used to express both chains. And in such cases, the two protein-coding sequences can be arranged in a head-to-tail orientation and can be connected with ribosome binding site (e.g., internal ribosomal binding site or IRES), protease cleavage site, or self-processing cleavage site (such as a sequence encoding a 2A peptide) to facilitate bicistronic expression. In some cases, the two chains can be linked with peptide linkers so that the two chains can be expressed as a single-chain polypeptide. Each expressed chain may contain the full variable domain sequence including the rearranged V(D)J gene. Each expressed chain may contain the full variable domain sequence including CDR1, CDR2, and CDR3. Each expressed chain may contain the full variable domain sequence including FR1, CDR1, FR2, CDR2, FR3, and CDR3. In some cases, each expressed chain may further contain a constant domain sequence.

To create expression vectors, additional sequences may need to be added to the fused TCR genes. These additional sequences include vector backbone (e.g., elements required for the vector's replication in target cell or in temporary host such as E. coli), promoters, IRES, sequence encoding the self-cleaving peptide, terminators, accessory genes (such as payloads), as well as partial sequences of the paired TCR-encoding polynucleotides (such as part of the sequences encoding the constant domains).

Protease cleavage sites include, but are not limited to, an enterokinase cleavage site: (Asp)4Lys (SEQ ID NO: 1); a factor Xa cleavage site: Ile-Glu-Gly-Arg (SEQ ID NO: 2); a thrombin cleavage site, e.g., Leu-Val-Pro-Arg-Gly-Ser (SEQ ID NO: 3); a renin cleavage site, e.g., His-Pro-Phe-His-Leu-Val-Ile-His (SEQ ID NO: 4); a collagenase cleavage site, e.g., X-Gly-Pro (where X is any amino acid); a trypsin cleavage site, e.g., Arg-Lys; a viral protease cleavage site, such as a viral 2A or 3C protease cleavage site, including, but not limited to, a protease 2A cleavage site from a picornavirus, a Hepatitis A virus 3C cleavage site, human rhinovirus 2A protease cleavage site, a picornavirus 3 protease cleavage site; and a caspase protease cleavage site, e.g., DEVD (SEQ ID NO: 5) recognized and cleaved by activated caspase-3, where cleavage occurs after the second aspartic acid residue. In some embodiments, the present disclosure provides an expression vector comprising a protease cleavage site, wherein the protease cleavage site comprises a cellular protease cleavage site or a viral protease cleavage site. In some embodiments, the first protein cleavage site comprises a site recognized by furin; VP4 of IPNV; tobacco etch virus (TEV) protease; 3C protease of rhinovirus; PC5/6 protease; PACE protease, LPC/PC7 protease; enterokinase; Factor Xa protease; thrombin; genenase I; MMP protease; Nuclear inclusion protein a (N1a) of turnip mosaic potyvirus; NS2B/NS3 of Dengue type 4 flaviviruses, NS3 protease of yellow fever virus; ORF V of cauliflower mosaic virus; KEX2 protease; CB2; or 2A. In some embodiments, the protein cleavage site is a viral internally cleavable signal peptide cleavage site. In some embodiments, the viral internally cleavable signal peptide cleavage site comprises a site from influenza C virus, hepatitis C virus, hantavirus, flavivirus, or rubella virus.

A suitable IRES element to include in the vector of the present disclosure can comprise an RNA sequence capable of engaging a eukaryotic ribosome. In some embodiments, an IRES element of the present disclosure is at least about 250 base pairs, at least about 350 base pairs, or at least about 500 base pairs. An IRES element of the present disclosure can be derived from the DNA of an organism including, but not limited to, a virus, a mammal, and a Drosophila. In some cases, a viral DNA from which an IRES element is derived includes, but is not limited to, picornavirus complementary DNA (cDNA), encephalomyocarditis virus (EMCV) cDNA and poliovirus cDNA. Examples of mammalian DNA from which an IRES element is derived includes, but is not limited to, DNA encoding immunoglobulin heavy chain binding protein (BiP) and DNA encoding basic fibroblast growth factor (bFGF). An example of Drosophila DNA from which an IRES element is derived includes, but is not limited to, an Antennapedia gene from Drosophila melanogaster. Addition examples of poliovirus IRES elements include, for instance, poliovirus IRES, encephalomyocarditis virus IRES, or hepatitis A virus IRES. Examples of flaviviral IRES elements include hepatitis C virus IRES, GB virus B IRES, or a pestivirus IRES, including but not limited to bovine viral diarrhea virus IRES or classical swine fever virus IRES.

Examples of self-processing cleavage sites include, but are not limited to, an intein sequence; modified intein; hedgehog sequence; other hog-family sequence; a 2A sequence, e.g., a 2A sequence derived from Foot and Mouth Disease Virus (FMDV); and variations thereof for each.

A vector for recombinant TCR expression may include any number of promoters, wherein the promoter is constitutive, regulatable or inducible, cell type specific, tissue-specific, or species specific. Further examples include tetracycline-responsive promoters. The vector can be a replicon adapted to the host cell in which the TCR is to be expressed, and it can comprise a replicon functional in a bacterial cell as well, for example, Escherichia coli. The promoter can be constitutive or inducible, where induction is associated with the specific cell type or a specific level of maturation, for example. Alternatively, a number of viral promoters can be suitable. Examples of promoters include the β-actin promoter, SV40 early and late promoters, immunoglobulin promoter, human cytomegalovirus promoter, retrovirus promoter, elongation factor 1A (EF-1A or EP-1alpha) promoter, phosphoglycerate kinase (PGK) promoter, and the Friend spleen focus-forming virus promoter. The promoters may or may not be associated with enhancers, wherein the enhancers may be naturally associated with the particular promoter or associated with a different promoter.

The recipient cell, which is the host cell for gene expression, can be, without limitation, an animal cell, especially a mammalian cell, or it can be a microbial cell (bacteria, yeast, or fungus) or a plant cell. Examples of host cells include insect cultured cells such as Spodoptera frugiperda cells, yeast cells such as Saccharomyces cerevisiae or Pichia pastoris, fungi such as Trichoderma reesei, Aspergillus, Aureobasidum and Penicillium species as well as mammalian cells such as CHO (Chinese hamster ovary), BHK (baby hamster kidney), COS, 293, 3T3 (mouse), Vero (African green monkey) cells and various transgenic animal systems, including without limitation, pigs, mice, rats, sheep, goat, cows, can be used as well. Baculovirus, especially AcNPV, vectors can be used for the single ORF TCR expression and cleavage of the present disclosure, for example with expression of the single ORF under the regulatory control of a polyhedrin promoter or other strong promoters in an insect cell line. Promoters used in mammalian cells can be constitutive (Herpes virus TK promoter; SV40 early promoter; Rous sarcoma virus promoter; cytomegalovirus promoter; mouse mammary tumor virus promoter) or regulated (metallothionein promoter, for example). Vectors can be based on viruses that infect particular mammalian cells, e.g., retroviruses, vaccinia and adenoviruses and their derivatives. Promoters include, without limitation, cytomegalovirus, adenovirus late, and the vaccinia 7.5K promoters. Enolase is an example of a constitutive yeast promoter, and alcohol dehydrogenase is an example of regulated promoter.

The selection of the specific promoters, transcription termination sequences and other optional sequences, such as sequences encoding tissue specific sequences, can be determined by the type of cell in which expression is carried out. The may be bacterial, yeast, fungal, mammalian, insect, chicken or other animal cells.

The TCR expressed from the TCR-expressing vectors may be in their natural form or may be in an engineered form. In some cases, the engineered form is a single-chain TCR fragment. In some cases, the engineered form is a TCR-CAR. Existing methods can also be used to introduce functional sequences (e.g., linkers, CD28 TM domains) to paired TCR-encoding polynucleotide in order to create TCR-expressing vectors that express these engineered forms of TCRs.

### Source TCR-Expressing Cells

The source TCR-expressing cells from which the two chains of a TCR can be informatically or physically paired may be of various cell types, from various organisms, and isolated from various tissues or organs. The source TCR-expressing cells can be obtained from various samples. The source TCR-expressing cells can produce TCRs. The source TCR-expressing cells may be immune cells. The immune cell refers to a cell of hematopoietic origin functionally involved in the initiation and/or execution of innate and/or adaptive immune response. The source TCR-expressing cells may be lymphocytes, e.g., tumor infiltrating lymphocytes (TILs). The source TCR-expressing cells may be a T cell.

The source TCR-expressing cells can be obtained or isolated from various samples. The source TCR-expressing cells can be immune cells obtained or isolated from various samples. The samples can be obtained from various sources or subjects described herein. The recipient cells may also be obtained from the samples described herein.

In certain embodiments, source TCR-expressing cells can be isolated from a blood sample or other biological samples of a subject or host, such as a human or other animal, such as a human or other animal that has been immunized or that is suffering from an infection, cancer, an autoimmune condition, or any other diseases to identify a pathogen-, tumor-, and/or disease specific antibody or TCR of potential clinical significance. For example, the human may be diagnosed with a disease, be exhibiting symptoms of a disease, not be diagnosed with a disease, or not be exhibiting symptoms of a disease. For example, the human may be one that was exposed to and/or who can make useful TCRs against an infectious agent (e.g., viruses, bacteria, parasites, prions, etc), antigen, or disease. For example, the animal may be one that was exposed to and/or who can make useful TCRs against an infectious agent (e.g., viruses, bacteria, parasites, prions, etc), antigen, or disease. Certain immune cells from immunized hosts can make TCRs to one or more target antigens in question and/or one or more unknown antigens. In the present disclosure the lymphocyte pool can be enriched for the desired immune cells by any suitable method, such as screening and sorting the cells using fluorescence-activated cell sorting (FACS), magnetic activated cell sorting (MACS), panning or other screening method to generate a plurality of immune cells from a sample.

The immune cell can be derived from a stem cell. The stem cells can be adult stem cells, embryonic stem cells, more particularly non-human stem cells, cord blood stem cells, progenitor cells, bone marrow stem cells, induced pluripotent stem cells, totipotent stem cells or hematopoietic stem cells. Representative human stem cells may be CD34+ cells. The isolated immune cell can be a dendritic cell, killer dendritic cell, a mast cell, a natural killer (NK) cell, a NK T cell, or a T cell selected from the group consisting of inflammatory T lymphocytes, cytotoxic T lymphocytes, regulatory T lymphocytes or helper T lymphocytes. The T cells can be CD4+T lymphocytes, CD8+T lymphocytes, or CD4+CD8+T lymphocytes.

In some embodiments, the source TCR-expressing cells can be immune cells isolated from non-immunized human or non-human donors. Immunizations may trigger any immune cell making a Va-Vβ or Vγ-Vδ combination that binds the immunogen to proliferate (clonal expansion). However, the use of spleen cells and/or immune cells or other peripheral blood lymphocytes from an unimmunized subject can provide a better representation of the possible TCR repertoire, and also permit the construction of a subsequent TCR library using any animal species.

In some cases, the source TCR-expressing cells can be obtained from peripheral blood sample. The peripheral blood cells can be enriched for a particular cell type (e.g., CD4+ cells; CD8+ cells; immune cells; T cells, or the like). The peripheral blood cells can also be selectively depleted of a particular cell type (e.g., mononuclear cells; red blood cells; CD4+ cells; CD8+ cells; immune cells; T cells, NK cells, or the like). A sample can comprise at least about 5, 10, 100, 250, 500, 750, 1000, 2500, 5000, 10000, 25000, 50000, 75000, 10000, 250000, 500000, 750000, 1000000, 2500000, 5000000, 7500000, or 10000000 subsets of or individual immune cells expressing different TCRs.

In some cases, the source TCR-expressing cells can be obtained from a tissue sample comprising a solid tissue, with non-limiting examples including a tissue from brain, liver, lung, kidney, prostate, ovary, spleen, lymph node (including tonsil), thyroid, thymus, pancreas, heart, skeletal muscle, intestine, larynx, esophagus, and stomach. Additional non-limiting sources include bone marrow, cord blood, tissue from a site of infection, ascites, pleural effusion, spleen tissue, and tumors. In some embodiments, T cell lines may be used. In some embodiments, the cell can be derived or obtained from a healthy donor, from a patient diagnosed with cancer or from a patient diagnosed with an infection. In some embodiments, the cell is part of a mixed population of cells which present different phenotypic characteristics.

The source TCR-expressing cells can be a tumor-infiltrating lymphocyte (TIL), e.g., tumor-infiltrating T cells. A TIL can be isolated from an organ afflicted with a cancer. One or more cells can be isolated from an organ with a cancer that can be a brain, heart, lungs, eye, stomach, pancreas, kidneys, liver, intestines, uterus, bladder, skin, hair, nails, ears, glands, nose, mouth, lips, spleen, gums, teeth, tongue, salivary glands, tonsils, pharynx, esophagus, large intestine, small intestine, rectum, anus, thyroid gland, thymus gland, bones, cartilage, tendons, ligaments, suprarenal capsule, skeletal muscles, smooth muscles, blood vessels, blood, spinal cord, trachea, ureters, urethra, hypothalamus, pituitary, pylorus, adrenal glands, ovaries, oviducts, uterus, vagina, mammary glands, testes, seminal vesicles, penis, lymph, lymph nodes or lymph vessels. One or more TILs can be from a brain, heart, liver, skin, intestine, lung, kidney, eye, small bowel, or pancreas. TILs can be from a pancreas, kidney, eye, liver, small bowel, lung, or heart. The one or more cells can be pancreatic islet cells, for example, pancreatic β cells. In some cases, a TIL can be from a gastrointestinal cancer. A TIL culture can be prepared a number of ways. For example, a tumor can be trimmed from non-cancerous tissue or necrotic areas. A tumor can then be fragmented to about 2-3 mm in length. In some cases, a tumor can be fragmented from about 0.5 mm to about 5 mm in size, from about 1 mm to about 2 mm, from about 2 mm to about 3 mm, from about 3 mm to about 4 mm, or from about 4 mm to about 5 mm. Tumor fragments can be enzymatically digested to obtain single-cell suspensions, from which lymphocytes or T cells can be isolated using existing methods. Tumor fragments can also be cultured in vitro utilizing media and a cellular stimulating agent such as a cytokine. In some cases, IL-2 can be utilized to expand TILs from a tumor fragment. A concentration of IL-2 can be about 6000 IU/mL. A concentration of IL-2 can also be about 2000 IU/mL, 3000 IU/mL, 4000 IU/mL, 5000 IU/mL, 6000 IU/mL, 7000 IU/mL, 8000 IU/mL, 9000 IU/mL, or up to about 10000 IU/mL. Once TILs are expanded, they can be subject to in vitro assays to determine tumor reactivity. For example, TILs can be evaluated by FACS for CD3, CD4, CD8, and CD58 expression. TILs can also be subjected to cocultured, cytotoxicity, ELISA, or ELISPOT assays. In some cases, TIL cultures can be cryopreserved or undergo a rapid expansion. A cell, such as a TIL, can be isolated from a donor of a stage of development including, but not limited to, fetal, neonatal, young and adult.

One or more samples can be from one or more sources. One or more of samples may be from two or more sources. One or more of samples may be from one or more subjects. One or more of samples may be from two or more subjects. One or more of samples may be from the same subject. One or more subjects may be from the same species. One or more subjects may be from different species. The one or more subjects may be healthy. The one or more subjects may be affected by a disease, disorder or condition.

A sample can be taken from a subject with a condition. In some embodiments, the subject from whom a sample is taken can be a patient, for example, a cancer patient or a patient suspected of having cancer. The subject can be a mammal, e.g., a human, and can be male or female. In some embodiments, the female is pregnant. The sample can be a tumor biopsy. The biopsy can be performed by, for example, a health care provider, including a physician, physician assistant, nurse, veterinarian, dentist, chiropractor, paramedic, dermatologist, oncologist, gastroenterologist, or surgeon.

The subject can have a disease in which a target antigen is expressed. For example, the disease can be cancer including, B-cell lymphoma, acute lymphoblastic leukemia (ALL), chronic lymphocytic leukemia, acute myeloid leukemia, adrenocortical carcinoma, adrenal cortex cancer, AIDS-related cancers, anal cancer, appendix cancer, astrocytomas, atypical teratoid/rhabdoid tumor, basal cell carcinoma, bile duct cancer, extrahepatic, bladder cancer, bone cancer (includes ewing sarcoma and osteosarcoma and malignant fibrous histiocytoma), brain tumors, breast cancer, burkitt lymphoma, carcinoid tumor (gastrointestinal), carcinoma of unknown primary, central nervous system, lymphoma, primary, cervical cancer, cholangiocarcinoma, chronic lymphocytic leukemia (cll), chronic myelogenous leukemia (cml), chronic myeloproliferative neoplasms, colorectal cancer, cutaneous t-cell lymphoma, ductal carcinoma in situ (dcis), endometrial cancer, esophageal, ewing sarcoma, extragonadal germ cell tumor, eye cancer, intraocular melanoma, retinoblastoma, fallopian tube cancer, fibrous histiocytoma of bone, malignant, and osteosarcoma, gallbladder cancer, gastric (stomach) cancer, gastrointestinal carcinoid tumor, gastrointestinal stromal tumors (gist), germ cell tumors, extragonadal, ovarian, testicular, gestational trophoblastic disease, gliomas, hairy cell leukemia, head and neck cancer, hepatocellular (liver) cancer, histiocytosis, langerhans cell, hodgkin lymphoma, hypopharyngeal cancer, intraocular melanoma, islet cell tumors, pancreatic neuroendocrine tumors, kaposi sarcoma, kidney, langerhans cell histiocytosis, laryngeal cancer, leukemia, lip and oral cavity cancer, liver cancer (primary), lung cancer, lymphoma, macroglobulinemia, waldenstrom, male breast cancer, malignant fibrous histiocytoma of bone and osteosarcoma, melanoma, melanoma, intraocular (eye), merkel cell carcinoma, mesothelioma, malignant, metastatic squamous neck cancer with occult primary, mouth cancer, multiple myeloma/plasma cell neoplasms, mycosis fungoides, myelodysplastic syndromes, myelodysplastic/myeloproliferative neoplasms and chronic myeloproliferative neoplasms, myelogenous leukemia, chronic (cml), myeloid leukemia, acute (AML), nasal cavity and paranasal sinus cancer, nasopharyngeal cancer, neuroblastoma, non-hodgkin lymphoma. non-small cell lung cancer, oral cancer, lip and oral cavity cancer and oropharyngeal cancer, osteosarcoma and malignant fibrous histiocytoma of bone, ovarian cancer, pancreatic cancer and pancreatic neuroendocrine tumors (islet cell tumors), paraganglioma, paranasal sinus and nasal cavity cancer, parathyroid cancer, penile cancer, pharyngeal cancer, pheochromocytoma, pituitary tumor, plasma cell neoplasm/multiple myeloma, pregnancy and breast cancer, primary central nervous system (CNS) lymphoma, primary peritoneal cancer, prostate cancer, rectal cancer, renal cell (kidney) cancer, retinoblastoma, salivary gland cancer, sarcoma, ewing sarcoma, kaposi sarcoma, osteosarcoma, rhabdomyosarcoma, uterine sarcoma, sézary syndrome, skin cancer, small cell lung cancer, small intestine cancer, soft tissue sarcoma, squamous cell carcinoma, squamous neck cancer with occult primary, metastatic, stomach (gastric) cancer, t-cell lymphoma, cutaneous, testicular cancer, throat cancer, thymoma and thymic carcinoma, thyroid cancer, transitional cell cancer of the renal pelvis and ureter, ureter and renal pelvis, transitional cell cancer, urethral cancer, uterine cancer, endometrial and uterine sarcoma, vaginal cancer, vulvar cancer, waldenstrom macroglobulinemia, or wilms tumor.

In some embodiments, a sample is a fluid, such as blood, saliva, lymph, urine, cerebrospinal fluid, seminal fluid, sputum, stool, or tissue homogenates. In some embodiments, the sample is saliva. In some embodiments, the sample is whole blood. In some embodiments, in order to obtain sufficient amount of cells, a blood volume of at least about 0.001, 0.005, 0.01, 0.05, 0.1, 0.5, 1, 2, 3, 4, 5, 10, 20, 25, 30, 35, 40, 45, or 50 mL is drawn. In some cases, blood can be collected into an apparatus containing a magnesium chelator including but not limited to EDTA, and is stored at 4° C. Optionally, a calcium chelator, including but not limited to EGTA, can be added. In some cases, a cell lysis inhibitor is added to the blood including but not limited to formaldehyde, formaldehyde derivatives, formalin, glutaraldehyde, glutaraldehyde derivatives, a protein cross-linker, a nucleic acid cross-linker, a protein and nucleic acid cross-linker, primary amine reactive crosslinkers, sulfhydryl reactive crosslinkers, sulfhydryl addition or disulfide reduction, carbohydrate reactive crosslinkers, carboxyl reactive crosslinkers, photoreactive crosslinkers, or cleavable crosslinkers. In some embodiments, non-nucleic acid materials can be removed from the starting material using enzymatic treatments (such as protease digestion).

A plurality of samples may comprise at least 2, 3, 4, 5, 10, 20, 30, 40, 50, 60, 70, 80, 90 or 100 or more samples. The plurality of samples may comprise at least about 100, 200, 300, 400, 500, 600, 700, 800, 900 or 1000 or more samples. The plurality of samples may comprise at least about 1000, 2000, 3000, 4000, 5000, 6000, 7000, 8000 samples, 9000, or 10,000 samples, or 100,000 samples, or 1,000,000 or more samples. The plurality of samples may comprise at least about 10,000 samples.

A first sample may comprise one or more cells and the second sample may comprise one or more cells. The one or more cells of the first sample may be of the same cell type as the one or more cells of the second sample. The one or more cells of the first sample may be of a different cell type as one or more different cells of the plurality of samples.

The plurality of samples may be obtained concurrently. A plurality of samples can be obtained at the same time. The plurality of samples can be obtained sequentially. A plurality of samples can be obtained over a course of years, e.g., 100 years, 10 years, 5 years, 4 years, 3 years, 2 years or 1 year of obtaining one or more different samples. One or more samples can be obtained within about one year of obtaining one or more different samples. One or more samples can be obtained within 12 months, 11 months, 10 months, 9 months, 8 months, 7 months, 6 months, 4 months, 3 months, 2 months or 1 month of obtaining one or more different samples. One or more samples can be obtained within 30 days, 28 days, 26 days, 24 days, 21 days, 20 days, 18 days, 17 days, 16 days, 15 days, 14 days, 13 days, 12 days, 11 days, 10 days, 9 days, 8 days, 7 days, 6 days, 5 days, 4 days, 3 days, 2 days or 1 day of obtaining one or more different samples. One or more samples can be obtained within about 24 hours, 22 hours, 20 hours, 18 hours, 16 hours, 14 hours, 12 hours, 10 hours, 8 hours, 6 hours, 4 hours, 2 hours or 1 hour of obtaining one or more different samples. One or more samples can be obtained within about 60 seconds, 45 seconds, 30 seconds, 20 seconds, 10 seconds, 5 seconds, 2 seconds or 1 second of obtaining one or more different samples. One or more samples can be obtained within less than one second of obtaining one or more different samples.

In some embodiments, TCRs from T cells of a particular phenotype are of interest. To achieve this, a subpopulation of T cells can be isolated/enriched/sorted. In one embodiment, a T cell population can be selected that expresses one or more of IFN-γ, TNF-alpha, IL-17A, IL-2, IL-3, IL-4, GM-CSF, IL-10, IL-13, granzyme B, and perform, or other appropriate molecules, e.g., other cytokines and transcription factors such as T-bet, Eomes, Tcf1 (TCF7 in human). Methods for screening for cell expression can be determined, e.g., by the methods described in PCT Publication No.: WO 2013/126712. In some embodiments, TCR sequences from T cell that also express one or more genes of interest, e.g., IFN-γ, TNF-alpha, IL-17A, IL-2, IL-3, IL-4, GM-CSF, IL-10, IL-13, granzyme B, perforin, T-bet, Eomes, Tcf1 (TCF7 in human) or other cytokines and transcription factors can be obtained by single-cell RNA sequencing.

T cells can be obtained from an in vitro culture. T cells can be activated or expanded in vitro by contacting with a tissue or a cell. For example, the T cells isolated from a patient's peripheral blood can be co-cultured with cells presenting tumor antigens such as tumor cells, tumor tissue, tumorsphere, tumor lysate-pulsed APC or tumor mRNA-loaded APC. The cells presenting tumor antigens may be APC pulsed with or engineered to express a defined antigen, a set of defined antigens or a set of undefined antigens (such as tumor lysate or total tumor mRNA). For example, in the cases of presenting defined antigens, an APC can express one or more minigenes encoding one or more short epitopes (e.g., from 7 to 60 amino acids in length) with known sequences. An APC can also express two or more minigenes from a vector containing sequences encoding the two or more epitopes. In the cases of presenting undefined antigens, an APC can be pulsed with tumor lysate or total tumor mRNA. The cells presenting tumor antigens may be irradiated before the co-culture. The co-culture may be in media comprising reagents (e.g., anti-CD28 antibody) that may provide co-stimulation signal or cytokines. Such co-culture may stimulate and/or expand tumor antigen-reactive T cells. These cells may be selected or enriched using cell surface markers described herein (e.g., CD25, CD69, CD137). Using this method, tumor antigen-reactive T cells can be pre-enriched from the peripheral blood of the patient, from TIL before expansion, or from TIL after expansion. These pre-enriched T cells can be used as the input to obtain fused (or physically linked) TCR using methods described herein or to obtained informatically paired TCR sequences using single-cell RNA-Seq. In some cases, the pre-enriched T cells may be used as the input to be subject to any other methods to identify the cognate pairs of the TCRs, for example, by sequencing using single cell barcodes. The antigen and APCs prepared using the methods described here can also be used to contact TCR-programmed recipient cells for various selection methods described elsewhere in this document.

When peripheral T cells are used in such in vitro culture, the pre-enriched T cells (e.g., CD137+) may contain T cells that acquired marker (e.g., CD137) expression during the co-culture, and may also contain T cells that already express the marker at blood draw. The latter population may nevertheless be tumor reactive. This method can offer an easier alternative to isolating TILs described.

In some cases, fresh tumor may not be available to isolate living TIL, and in such cases, nuclei may be isolated from frozen or fixed tissue. These nuclei may also serve as the input for the paired bipartite immunoreceptor cloning process (in this case paired TCR cloning) to obtain fused TCR polynucleotide library or as the input for single-cell RNA Seq to obtained informatically paired TCR sequences.

### Diversity of TCRs

The original site that hosts the source TCR-expressing cells may affect the characteristics of the repertoire of the paired TCR-encoding polynucleotides, and consequently, the repertoire of the resultant TCR-expressing vectors and the TCR-programmed recipient cells. An aspect of the characteristics of such repertoire can be the gene usage diversity.

In some cases, the repertoire may contain more than 2, more than 5, more than 10, more than 50, more than 100, more than 500, more than 1,000, more than 5,000, more than 10,000, more than 50,000, or more than 100,000 V(D)J combinations. In some cases, the repertoire may contain more than 2, more than 5, more than 10, more than 50, more than 100, more than 500, more than 1,000, more than 5,000, more than 10,000, more than 50,000, or more than 100,000 different V(D)J combinations. This is because the polyclonal population of the source TCR-expressing cells may have highly diverse usage of V, D, and J genes, resulting in highly diverse V(D)J combinations.

A VJ combination of a paired TCR-encoding polynucleotide or a TCR-expressing vector may be defined by the V gene and J gene used by both TCR chains. A V(D)J combination of a paired TCR-expressing polynucleotide or a TCR-expressing vector may be defined by the V gene, D gene, and J gene used by both TCR chains. For example, TRAV8-4/TRAJ45/TRBV29-1/TRBJ1-5 can define a particular VJ recombination of a paired TCR. Given a coding sequence for a TCR chain, one may deduce the V(D)J combination using computational tools such as V-Quest and MiXCR.

It should be noted that two different paired TCR-expressing polynucleotides (or two different immunoreceptor-expressing vectors) may share the same V(D)J recombination but have different sequences, this may be because (1) during the V(D)J recombination random insertion and deletions may happen at the V-D, D-J, and V-J junctions, and (2) sequence variations may be artificially created by mutagenesis and gene synthesis, and variable sequences may be introduced during the gene synthesis. For example, two fused TCR genes may have the same VJ recombination but have different CDR3 sequences. The paired TCR-expressing polynucleotide or expression vector can comprise a cognate pair combination (or a native pair combination in a cell) of a first TCR chain and a second TCR chain from a source TCR-expressing cell. A plurality of paired TCR-expressing polynucleotides or expression vectors can comprise multiple cognate pair combinations of first TCR chains and second TCR chains from a plurality of TCR-expressing cells. The source TCR-expressing cells can have different clonotypes, and therefore can result in a polyclonal population of paired TCR-expressing polynucleotides or expression vectors. Delivering the polyclonal TCR-expressing vectors into a plurality of recipient cell can produce a polyclonal population of TCR-programmed recipient cells, expressing at least 100, at least 1,000, at least 10,000, at least 100,000, at least 1,000,000, at least 10,000,000, or at least 100,000,000, or more different TCRs (or different cognate pair combinations of TCRs). Each of the different TCRs can have a unique sequence in the paired TCR-expressing polynucleotide.

This polyclonal feature can distinguish the library of paired TCR-expressing polynucleotides, the library of expression vectors and the polyclonal population of TCR-programmed recipient cells obtained using methods described in this disclosure from previously reported counterparts. For example, using existing methods one may start with one or a handful of TCR-coding sequences, TCR domain-coding sequences, or paired TCR-expressing polynucleotides, and generate a large number of variations of these starting sequences using mutagenesis or error prone PCR to create a large library of paired TCR-expressing polynucleotides. Thus, these libraries may contain one or a handful of V(D)J gene combinations. In contrast, the library of paired TCR-expressing polynucleotides, the library of expression vectors and the polyclonal population of TCR-programmed recipient cells obtained using methods described in this disclosure can contain more than about 1,000, more than about 5,000, more than about 10,000, more than about 50,000, more than about 100,000, more than about 500,000, more than about 1,000,000, more than about 5,000,000, or more than about 10,000,000 sequences and may contain more than about 1,000, more than about 5,000, more than about 10,000, more than about 50,000, more than about 100,000, more than about 500,000, more than about 1,000,000, more than about 5,000,000, or more than about 10,000,000 VJ or VDJ combinations. In some cases, the library of paired TCR-expressing polynucleotides, the library of expression vectors and the polyclonal population of TCR-programmed recipient cells obtained using methods described in this disclosure can contain at least about 50, at least about 100, at least about 200, at least about 500, at least about 1,000, at least about 2,000, at least about 5,000, at least about 10,000, at least about 100,000, at least about 1,000,000, or at least about 10,000,000 VJ or VDJ combinations. Moreover, the library of paired TCR-expressing polynucleotides, the library of expression vectors and the polyclonal population of immunoreceptor-programmed recipient cells obtained using methods described in this disclosure may contain at least 10, at least 15, at least 20, or more different TRAV (V gene for TCRα chain) subgroups, and/or at least 10, at least 15, at least 20, or more different TRBV (V gene for TCRβ chain) subgroups.

### Source of Recipient Cells

The recipient cells (e.g., TCR-programmed recipient cells) can be from various sources. The recipient cells can be T cells. T cells used as recipient cells can be obtained from a subject (e.g., primary T cells). In some cases, T cells as the source TCR-expressing cells are obtained from a subject. T cells may be obtained from any sample described herein. In some cases, the recipient T cells are obtained from a subject. The term “subject” refers to an organism. For example, the organism can be a living organism in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain aspects, T cell lines may be used. T cells can be helper T cells, a cytotoxic T cells, memory T cells, regulatory T cells, natural killer T cells, alpha beta T cells, or gamma delta T cells. In certain aspects of the present disclosure, T cells can be obtained from a unit of blood collected from a subject using a variety of techniques, such as Ficoll™ separation. Cells from the circulating blood of an individual can be obtained by apheresis. The apheresis product may contain lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing steps. In some cases, the cells can be washed with phosphate buffered saline (PBS). The wash solution may lack calcium or magnesium or other divalent cations. Initial activation steps in the absence of calcium can lead to magnified activation. A washing step may be accomplished by methods such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In an aspect, T cells are isolated from peripheral blood lymphocytes or tissues by lysing the red blood cells and depleting the monocytes, for example, by centrifugation through a PERCOLL™ gradient or by counterflow centrifugal elutriation. When isolating T cells from tissues (e.g., isolating tumor-infiltrating T cells from tumor tissues), the tissues made be minced or fragmented to dissociate cells before lysing the red blood cells or depleting the monocytes. A specific subpopulation of T cells, such as CD3+, CD28+, CD4+, CD8+, CD45RA+, and CD45RO+ T cells, can be further isolated by positive or negative selection techniques. For example, T cells can be isolated by incubation with anti-CD3/anti-CD28 (e.g., 3×28)-conjugated beads, such as DYNABEADS™ M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least or equal to about 1, 2, 3, 4, 5, or 6 hours. In yet another aspect, the time period is 10 to 24 hours. In an aspect, the incubation time period is about 24 hours. Longer incubation times may be used to isolate T cells in any situation where there are few T cells as compared to other cell types, such as in isolating tumor infiltrating lymphocytes (TILs) from tumor tissue or from immunocompromised individuals. Further, use of longer incubation times can increase the efficiency of capture of CD8+ T cells. Thus, by simply shortening or lengthening the time T cells are allowed to bind to the anti-CD3/anti-CD28 beads and/or by increasing or decreasing the ratio of beads to T cells, subpopulations of T cells can be selected for or against at culture initiation or at other time points during the process. Additionally, by increasing or decreasing the ratio of anti-CD3 and/or anti-CD28 antibodies on the beads or other surface, subpopulations of T cells can be selected for or against at culture initiation or at other desired time points. In some cases, multiple rounds of selection can be used. In certain aspects, the selection procedure can be performed and the “unselected” cells (cells that may not bind to the anti-CD3/anti-CD28 beads) can be used in the activation and expansion process. “Unselected” cells can also be subjected to further rounds of selection.

Enrichment of a T cell population by negative selection can be accomplished with a combination of antibodies directed to surface markers unique to the negatively selected cells. One example method is cell sorting and/or selection via negative magnetic immune adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be useful to enrich for or positively select for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

In one embodiment, a T cell population can be selected that expresses one or more of IFN-γ, TNF-alpha, IL-17A, IL-2, IL-3, IL-4, GM-CSF, IL-10, IL-13, granzyme B, and perforin, or other appropriate molecules, e.g., other cytokines and transcription factors such as T-bet, Eomes, Tcf1 (TCF7 in human). Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, the volume in which beads and cells are mixed together may be decreased (e.g., increase the concentration of cells) to ensure maximum contact of cells and beads. For example, in an aspect, a concentration of 2 billion cells/mL is used. In another aspect, a concentration of 1 billion cells/mL is used. In a further aspect, greater than 100 million cells/mL is used. In a further aspect, a concentration of cells of at least about 10, 15, 20, 25, 30, 35, 40, 45, or 50 million cells/mL is used. In some aspects, a concentration of cells of at least about 75, 80, 85, 90, 95, or 100 million cells/mL is used. In some aspects, a concentration of cells of at least about 125 or 150 million cells/mL can be used. Using high concentrations can result in increased cell yield, cell activation, and cell expansion. Further, use of high cell concentrations can allow more efficient capture of cells that may weakly express target antigens of interest, such as CD28-negative T cells, or from samples where there are many tumor cells present (e.g., leukemic blood, tumor tissue, etc.). Such populations of cells may have therapeutic value. For example, using high concentration of cells allows more efficient selection of CD8+ T cells that may have weaker CD28 expression.

In some cases, lower concentrations of cells may be used. By significantly diluting the mixture of T cells and surface, interactions between the particles and cells can be minimized. This can select for cells that express high amounts of desired antigens to be bound to the particles. For example, CD4+ T cells can express higher levels of CD28 and can be more efficiently captured than CD8+ T cells in dilute concentrations. In some aspects, the concentration of cells used is at least about 5×10⁵/mL, 5×10⁶/mL, or more. In other aspects, the concentration used can be from about 1×10⁵/mL to 1×10⁶/mL, and any integer value in between. In other aspects, the cells may be incubated on a rotator for varying lengths of time at varying speeds at either 2-10° C. or at room temperature.

T cells for stimulation can also be frozen after a washing step. The freeze and subsequent thaw step may provide a more uniform product by removing granulocytes and to some extent monocytes in the cell population. After the washing step that removes plasma and platelets, the cells may be suspended in a freezing solution. While many freezing solutions and parameters may be useful in this context, one method that can be used involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing Hespan and PlasmaLyte A. The cells can then be frozen to −80° C. and stored in the vapor phase of a liquid nitrogen storage tank. Cell may be frozen by uncontrolled freezing immediately at −20° C. or in liquid nitrogen. In certain aspects, cryopreserved cells are thawed and washed as described herein and allowed to rest for one hour at room temperature prior to activation.

Also contemplated in the context of the present disclosure is the collection of blood samples or apheresis product from a subject at a time period prior to when expanded cells (e.g., engineered cells expressing TCRs for T cell therapy) might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In some cases, a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present disclosure, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Thus, it is contemplated within the context of the present disclosure to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

Besides primary T cells obtained from a subject, the T cells used as recipient cell may be cell-line cells, such as cell-line T cells. Examples of cell-line T cells include, but are not limited to, Jurkat, CCRF-CEM, HPB-ALL, K-T1, TALL-1, MOLT 16/17, and HUT 78/H9.

The recipient cells can be T cells, B cells, NK cells, macrophages, neutrophils, granulocytes, eosinophils, red blood cells, platelets, stem cells, iPSCs, or mesenchymal stem cells. In addition, the recipient cell can be a cell line cell. The cell line can be tumorigenic or artificially immortalized cell line. Examples of cell lines include, but are not limited to, CHO-K1 cells; HEK293 cells; Caco2 cells; U2-OS cells; NIH 3T3 cells; NSO cells; SP2 cells; CHO-S cells; DG44 cells; K-562 cells, U-937 cells; MRCS cells; IMR90 cells; Jurkat cells; HepG2 cells; HeLa cells; HT-1080 cells; HCT-116 cells; Hu-h7 cells; Huvec cells; and Molt 4 cells. The recipient cell can be an autologous T cell or an allogeneic T cell. The recipient cell can be a genetically modified or engineered cell. In some embodiments, the recipient cell expresses proteins required to form functional TCR complex (e.g., CD3-gamma, CD3-delta, CD3-epsilon, CD3-zeta). In these cells, an exogenous TCR can be expressed in its natural form. In some embodiments, the recipient cell does not express all proteins required to form functional TCR complex. In these cells an exogenous TCR can be expressed in an engineered form. In some cases, the engineered form is a single-chain TCR fragment. In some cases, the engineered form is a TCR-CAR.

### Preparation of Antigen Presenting Cells (APCs)

Source TCR-expressing cells or TCR-programmed recipient cells may be co-cultured with an antigen presenting cell (APC) so that the antigen presented by APC may contact the TCR expressed by the TCR-expressing cells or TCR-programmed recipient cells (which can be T cells). The APC may be an artificial APC (aAPC). The APC can be professional APC such as dendritic cell, macrophage, or B cell. The APC can be a monocyte or monocyte-derived dendritic cell. An aAPC can express ligands for TCR and costimulatory molecules and can activate and expand T cells. An aAPC can be engineered to express any gene for T cell activation. An aAPC can be engineered to express any gene for T cell expansion. An aAPC can be a bead, a cell, a protein, an antibody, a cytokine, or any combination. An aAPC can deliver signals to a cell population that may undergo genomic transplant. For example, an aAPC can deliver a signal 1, signal, 2, signal 3 or any combination. A signal 1 can be an antigen recognition signal. For example, signal 1 can be ligation of a TCR by a peptide-MHC complex or binding of agonistic antibodies directed towards CD3 that can lead to activation of the CD3 signal-transduction complex. Signal 2 can be a co-stimulatory signal. For example, a co-stimulatory signal can be anti-CD28, inducible co-stimulator (ICOS), CD27, and 4-1BB (CD137), which bind to ICOS-L, CD70, and 4-1BBL, respectively. Signal 3 can be a cytokine signal. A cytokine can be any cytokine. A cytokine can be IL-2, IL-7, IL-12, IL-15, IL-21, or any combination thereof.

In some cases, an aAPC may be used to stimulate, activate and/or expand a cell population. In some cases, an aAPC may not induce allospecificity. An aAPC may not express HLA in some cases. An aAPC may be genetically modified to stably express genes that can be used to activation and/or stimulation. In some cases, a K562 cell may be used for activation. A K562 cell may also be used for expansion. A K562 cell can be a human erythroleukemic cell line. A K562 cell may be engineered to express genes of interest. K562 cells may not endogenously express HLA class I, II, or CD1d molecules but may express ICAM-1 (CD54) and LFA-3 (CD58). K562 may be engineered to deliver a signal 1 to T cells. For example, K562 cells may be engineered to express HLA class I. In some cases, K562 cells may be engineered to express additional molecules such as B7, CD80, CD83, CD86, CD32, CD64, 4-1BBL, anti-CD3, anti-CD3 mAb, anti-CD28, anti-CD28mAb, CD1d, anti-CD2, membrane-bound IL-15, membrane-bound IL-17, membrane-bound IL-21, membrane-bound IL-2, truncated CD19, or any combination. In some cases, an engineered K562 cell can expresses a membranous form of anti-CD3 mAb, clone OKT3, in addition to CD80 and CD83. In some cases, an engineered K562 cell can expresses a membranous form of anti-CD3 mAb, clone OKT3, membranous form of anti-CD28 mAb in addition to CD80 and CD83.

In some cases, stimulation of T cells can be performed with antigen and irradiated, histocompatible APCs, such as feeder PBMCs. In some cases, cells can be grown using non-specific mitogens such as PHA and allogenic feeder cells. Feeder PBMCs can be irradiated at 40Gy. Feeder PBMCs can be irradiated from about 10 Gy to about 15 Gy, from about 15 Gy to about 20 Gy, from about 20Gy to about 25 Gy, from about 25 Gy to about 30 Gy, from about 30 Gy to about 35 Gy, from about 35 Gy to about 40 Gy, from about 40 Gy to about 45 Gy, from about 45 Gy to about 50 Gy. In some cases, a control flask of irradiated feeder cells only can be stimulated with anti-CD3 and IL-2.

An aAPC can be a bead. A spherical polystyrene bead can be coated with peptide-MHC complex and optionally antibodies against CD28 and be used for T cell activation or stimulation. A bead can be of any size. In some cases, a bead can be or can be about 3 and 6 micrometers. A bead can be or can be about 4.5 micrometers in size. A bead can be utilized at any cell to bead ratio. For example, a 3 to 1 bead to cell ratio at 1 million cells per milliliter can be used. An aAPC can also be a rigid spherical particle, a polystyrene latex microbeads, a magnetic nano- or micro-particles, a nanosized quantum dot, a poly(lactic-co-glycolic acid) (PLGA) microsphere, a nonspherical particle, a carbon nanotube bundle, a ellipsoid PLGA microparticle, a nanoworms, a fluidic lipid bilayer-containing system, a 2D-supported lipid bilayer (2D-SLBs), a liposome, a RAFTsomes/microdomain liposome, an SLB particle, or any combination thereof.

In some cases, an aAPC can expand CD4 T cells. For example, an aAPC can be engineered to mimic an antigen processing and presentation pathway of HLA class II-restricted CD4 T cells. A K562 can be engineered to express HLA-D, DP α, DP β chains, Ii, DM α, DM β, CD80, CD83, or any combination thereof. For example, engineered K562 cells can be pulsed with an HLA-restricted peptide in order to expand HLA-restricted antigen-specific CD4 T cells.

In some cases, the use of aAPCs can be combined with exogenously introduced cytokines for T cell activation, expansion, or any combination. Cells can also be expanded in vivo, for example in the subject's blood after administration of genomically transplanted cells into a subject.

### Identification of Putative Antigen-Reactive TCRs

Methods provided herein can be used to identify antigen-reactive TCRs from T cells. T cells (e.g., source TCR-expressing cells) can be screened from multiple organs such as peripheral blood, spleen, lymph node, and tumor in order to identify TCRs that recognize a particular or a particular population of MHC-bound antigen. The polyclonal TCR-programmed recipient cells obtained using methods described herein can replace the source TCR-expressing cells in these applications. In these applications, the recipient cells may be cell-line cells, such as cell-line T cells. Examples of cell-line T cells include, but are not limited to, Jurkat, CCRF-CEM, HPB-ALL, K-T1, TALL-1, MOLT 16/17, and HUT 78/H9. The endogenous TCR of the cell-line T cells may be inactivated (e.g., knocked out or knocked down). For example, the method provided herein can comprise providing a plurality of T cells expressing a plurality of TCRs (e.g., source TCR-expressing cells), where each T cell of the plurality of T cells can expresse a cognate pair of a TCR of the plurality of TCRs. Next, a first polynucleotide encoding a first TCR chain and a second polynucleotide encoding a second TCR chain of the cognate pair of the TCR of each T cell can be paired, thereby generating a plurality of polynucleotide pairs. Polynucleotides comprising the plurality of polynucleotide pairs can be delivered into a plurality of recipient cells (e.g., TCR-programmed recipient cells), where each recipient cell comprises a polynucleotide comprising at least one polynucleotide pair of the plurality of polynucleotide pairs. The polynucleotides can be the plurality of polynucleotide pairs or copies of the plurality of polynucleotide pairs. The polynucleotides can be vectors (e.g., expression vectors described herein) comprising sequences of the plurality of polynucleotide pairs. The plurality of polynucleotide pairs can be expressed in the plurality of recipient cells. The TCR repertoire of the plurality of recipient cells can be sequenced and a frequency of a TCR of the TCR repertoire in the plurality of recipient cells can be determined. Next, the plurality of recipient cells can be contacted with one or more antigens. Upon contacting, a marker can be activated on a subset of the plurality of recipient cells, and the subset of the plurality of recipient cells can be isolated based on the marker. The TCR repertoire of the subset of the plurality of recipient cells can be sequenced and a frequency of a TCR of the TCR repertoire in the subset of the plurality of recipient cells can be determined. The TCR having a frequency in the subset of the plurality of recipient cells that is higher than its frequency in the plurality of recipient cells can be identified as the putative target-reactive TCR.

The antigen described herein can be a MHC-bound antigen. In some embodiments, the MHC-bound antigen is a peptide MHC complex (pMHC), pMHC tetramer, pMHC oligomer. For example, pMHC can be tetramerized on a streptavidin scaffold, or oligomerized on a variety of chemical scaffolds. In some embodiments, the pMHC, pMHC tetramer, pMHC oligomer is fluorescently labeled to facilitate FACS sorting of polyclonal TCR-programmed recipient cells that recognize the pMHC. In some embodiments, the pMHC, pMHC tetramer, pMHC oligomer can be bound to a solid surface to facilitate the enrichment of polyclonal TCR-programmed recipient cells that recognize the pMHC. The surface solid surface may be the solid surface of a particle, a nanoparticle, a magnetic particle or a magnetic nanoparticle. In some embodiments, the MHC-bound antigen is presented on the surface of a cell. In some cases, the cell is an antigen presenting cell (APC). The APC can be professional APC such as dendritic cell, macrophage, or B cell. The APC may also be other cells (e.g., artificial APC) expressing MHC or HLA. For example, a cell from a cancer cell line can be APC. In some embodiments, the APC can be engineered to express only one Class I MHC allele. In some embodiments, the APC may be engineered to express an arbitrary number of Class I MHC alleles and Class II MHC alleles such as all the Class I or Class II MHC alleles isolated from one subject. The subject may be a human. The human may be a patient. The patient may be a cancer patient.

In some embodiments, the epitope of MHC-bound antigen is well defined. For example, in pMHC tetramer the epitope peptide can be chemically synthesized. In some embodiments, the epitope for the MHC-bound antigen is unknown or not well defined. For example, an antigen protein can be over-expressed in the APC, and multiple epitopes may be presented by the APC. In another example, a small group of proteins (e.g., at least 2 proteins, at least 3 proteins, at least 4 proteins, at least 5 proteins, at least 10 proteins, at least 20 proteins, at least 30 proteins, at least 40 proteins, or at least 50 proteins) can be over-expressed in the APC. In some cases, a fragment of the antigen encompassing the possible epitope can be over-expressed in APC. For example, the antigen may be a polypeptide encoding part of a mutant protein, wherein the mutated residue is approximately in the middle of the polypeptide. The antigen may also be a polypeptide encoding part of an abnormally expressed protein. Such polypeptide is sometimes referred to synthetic long peptide (SLP). A polynucleotide encoding such polypeptide is sometimes referred to as minigene. A series of 5 to 20 minigenes can be serially linked with linker peptide in a long polypeptide chain, which is sometimes referred to as tandem minigene (TMG). The minigene can be DNA or RNA and can be in a number of format (e.g., plasmid, viral vector, mRNA, RNA replicon). A pool of minigenes can be introduced to APC by methods such as transfection, transformation, transduction, and electroporation, depending on the format of the minigene. The minigene can encode a mutated peptide (e.g., with point mutation) or a peptide that is over-expressed in tumors due to other genetic or epigenetic abnormalities, such as fusion, deletion, insertion, frameshift, intron inclusion, alternative splicing, or over-expression. Such peptides can be identified in tumor sample through state-of-the-art genomic, transcriptomic, and/or proteomic analyses such as whole exome sequencing (WES), whole genome sequencing (WGS), transcriptome sequencing (RNA-Seq), MHC-peptide elution mass spectrometry, or a combination thereof. In another example, an unknown number of proteins can be over-expressed in the APC, and in such cases, a cDNA pool can be delivered (e.g., transfected, electroporated, or other delivery methods using a vector described herein) into the APC.

In some embodiments, the antigen can be introduced to the APC by transfecting the antigen-coding DNA or mRNA into the APC. In some embodiments, the antigen as proteins may be added to the culture media of the APC. In some embodiments, the antigen as peptides may be added to the culture media of the APC.

The antigen can be a target antigen. The antigen can be a tumor antigen. The antigen can include a tumor-specific antigen, a tumor-associated antigen, a cell that expresses tumor-specific antigens, a cell that expresses tumor-associated antigen, an embryonic antigen on tumor, an autologous tumor cell, a tumor-specific membrane antigen, a tumor-associated membrane antigen, a growth factor receptor, a growth factor ligand, or any other type of antigen or antigen-presenting cell or material that is associated with a cancer. The tumor antigen can be a tumor-specific antigen (TSA). The term “TSA,” as used herein, refers to an antigen that is unique to tumor cells and does not occur on other cells in the body. The tumor antigen can be a tumor-associated antigen (TAA). The term “TAA,” as used herein, refers to an antigen that is not unique to a tumor cell and is also expressed on a normal cell. The expression of the antigen on the tumor can occur under conditions that enable the immune system to respond to the antigen. The TAA may be expressed at much higher levels on tumor cells. The TAA can be determined by sequencing a patient's tumor cells and identifying mutated proteins only found in the tumor. These antigens are referred to as “neoantigens.” The tumor antigen can be an epithelial cancer antigen, a prostate specific cancer antigen (PSA) or prostate specific membrane antigen (PSMA), a bladder cancer antigen, a lung cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, a colorectal cancer antigen, a lymphoma antigen, a B-cell lymphoma cancer antigen, a leukemia antigen, a myeloma antigen, an acute lymphoblastic leukemia antigen, a chronic myeloid leukemia antigen, or an acute myelogenous leukemia antigen. Examples of antigens include, but are not limited to, 1GH-IGK, 43-9F, 5T4, 791Tgp72, 9D7, acyclophilin C-associated protein, alpha-fetoprotein (AFP), α-actinin-4, A3, antigen specific for A33 antibody, ART-4, B7, Ba 733, BAGE, BCR-ABL, beta-catenin, beta-HCG, BrE3-antigen, BCA225, BING-4, BRCA1/2, BTAA, CA125, CA 15-3\CA 27.29\BCAA, CA195, CA242, CA-50, calcium activated chloride channel 2, CAGE, CAM43, CAMEL, CAP-1, carbonic anhydrase IX, c-Met, CA19-9, CA72-4, CAM 17.1, CASP-8/m, CCCL19, CCCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD68, CD70, CD70L, CD74, CD79a, CD79b, CD80, CD83, CD95, CD126, CD132, CD133, CD138, CD147, CD154, CDC27, CDK4, CDK4m, CDKN2A, CML6/6, CO-029, CTLA4, CXCR4, CXCR7, CXCL12, cyclin B, HIF-1a, colon-specific antigen-p (CSAp), CEA (CEACAMS), CEACAM6, c-Met, DAM, E2A-PRL, EGFR, EGFRvIII, EGP-1 (TROP-2), EGP-2, ELF2-M, Ep-CAM, EphA3, fibroblast growth factor (FGF), FGF-5, fibronectin, Flt-1, Flt-3, folate receptor, G250 antigen, Ga733VEpCAM, GAGE, gp100, GRO-β, H4-RET, HLA-DR, HM1.24, human chorionic gonadotropin (HCG) and its subunits, HMGB-1, hypoxia inducible factor (HIF-1), HSP70-2M, HST-2, HTgp-175, Ia, IGF-1R, IFN-γ, IFN-α, IFN-β, IFN-k, IL-4R, IL-6R, IL-13R, IL-15R, IL-17R, IL-18R, IL-2, IL-6, IL-8, IL-12, IL-15, IL-17, IL-18, IL-23, IL-25, immature laminin receptor, insulin-like growth factor-1 (IGF-1), KC4-antigen, KSA, KS-1-antigen, KS1-4, LAGE-1a, Le-Y, LDR/FUT, M344, MA-50, macrophage migration inhibitory factor (MIF), MAGE, MAGE-1, MAGE-3, MAGE-4, MAGE-5, MAGE-6, MART-1, MART-2, TRAG-3, MC1R, mCRP, MCP-1, mesothelin, MIP-1A, MIP-1B, MIF, MG7-Ag, MOV18, MUC1, MUC2, MUC3, MUC4, MUCSac, MUC13, MUC16, MUM-1/2, MUM-3, MYL-RAR, NB/70K, Nm23H1, NuMA, NCA66, NCA95, NCA90, NY-ESO-1, P polypeptide, p15, p16, p53, p185erbB2, p180erbB3, PAM4 antigen, pancreatic cancer mucin, PD1 receptor (PD-1), PD-1 receptor ligand 1 (PD-L1), PD-1 receptor ligand 2 (PD-L2), PI5, placental growth factor, p53, PLAGL2, Pme117 prostatic acid phosphatase, PSA, PRAME, PSMA, P1GF, ILGF, ILGF-1R, IL-6, IL-25, RCAS1, RS5, RAGE, RANTES, Ras, T101, SAGE, SAP-1, 5100, SSX-2, survivin, survivin-2B, SDDCAG16, TA-90\Mac2 binding protein, TAAL6, TAC, TAG-72, TGF-βRII, Ig TCR, TLP, telomerase, tenascin, TRAIL receptors, TRP-1, TRP-2, TSP-180, TNF-α, Tn antigen, Thomson-Friedenreich antigens, tumor necrosis antigens, tyrosinase, VEGFR, ED-B fibronectin, WT-1, XAGE, 17-1A-antigen, complement factors C3, C3a, C3b, C5a, C5, an angiogenesis marker, bcl-2, bcl-6, and K-ras, an oncogene marker and an oncogene product.

The TCR-programmed recipient cells that recognize the MHC-bound antigen may be selected from those that do not. When the TCR-programmed recipient cells are used for this purpose (e.g., enriching or identifying TCRs that recognize an antigen) they can also be called TCR-programmed reporter cells or TCR-engineered reporter cells. The selection may be based on binding to soluble, fluorescently labeled, or surface-bound pMHC, pMHC tetramer or pMHC oligomer. The selection may be based on marker (e.g., T cell activation marker) expression on the TCR-programmed recipient cells after the cells contact MHC-bound antigen. The marker may be a cell surface marker. The cell surface marker may be CD39, CD69, CD103, CD25, PD-1, TIM-3, OX-40, 4-1BB, CD137, CD3, CD28, CD4, CD8, CD45RA, CD45RO, GITR, FoxP3, as well as other T cell activation markers, or a combination thereof. The selection may be based on calcium influx. The marker may be intracellular protein or a secreted protein. The intracellular protein may be a transcription factor or may be a phosphorylated protein. The secreted protein may be a cytokine or a chemokine (e.g., IFN-γ, TNF-alpha, IL-17A, IL-2, IL-3, IL-4, GM-CSF, IL-10, IL-13, granzyme B, perforin, or a combination thereof). When using a secreted protein as the marker, inhibitors of protein trafficking may be added to the cell. The inhibitor of protein trafficking may be a Golgi blocker. The Golgi blocker may be Brefeldin A, Monensin or the like. The secreted protein may be IL-2, IL-10, IL-15, TNF-alpha, or INF-gamma. The selection may also be based on reporter gene expression or a reporter protein. The reporter protein may be a fluorescent protein (such as GFP and mCherry). The reporter gene expression may be under the control of a transcription factor which is regulated by TCR signaling. Examples of these transcription factors include, but are not limited to, AP-1, NFAT, NF-kappa-B, Runx1, Runx3, etc. As described herein, in some cases, prior to selection, the pre-selection pool of TCR-programmed recipient cells can comprise at least about 0.001%, 0.01%, 0.1%, 0.2%, 0.5%, 0.8%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, or more target-reactive TCRs. In some cases, the pre-selection pool of TCR-programmed recipient cells can comprise at most about 30%, 25%, 20%, 15%, 10%, 5%, 3%, 1%, 0.5%, 0.1%, 0.01%, or less target-reactive TCRs. After selection, the percentage of target-reactive TCRs can be enriched in the post-selection pool of TCR-programmed recipient cells. The post-selection pool of TCR-programmed recipient cells can comprise at least about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 15%, 20%, 25%, 30%, 35%, 40%, 45%, 50%, 55%, or more target-reactive TCRs.

In some embodiments, the selected TCR-programmed recipient cells based on the criteria described above can be propagated and undergo selection again in order to further enrich the TCRs that recognize the MHC-bound antigen. In some embodiments, the fused TCR polynucleotides in the TCR-expressing vectors isolated from the selected TCR-programmed recipient cells can be amplified and converted into TCR-expressing vectors. And these TCR-expressing vectors can be used to obtain a new population of TCR-programmed recipient cells. These cells can undergo selection again in order to further enrich the TCRs that recognize the MHC-bound antigen.

Rapid identification of tumor-reactive TCRs without necessarily knowing the identity of the antigen, epitope or the presenting MHC can have broad applications in cancer immunotherapy, and can be achieved by the massively parallel TCR cloning technologies or high throughput TCR synthesis technologies described herein combined with a reporter-based selection method.

An example scheme of the reporter-based selection method is outlined below. The reporter cell line can be a T cell line (e.g., Jurkat). The reporter cell line may carry a reporter gene (e.g., a florescent protein or a stainable cell surface protein) driven by a promoter controlled by TCR signaling (e.g., NFAT, NF-kappa-B, Nur77). Optionally, the endogenous TCR of the reporter cell line can be knocked out. The reporter cells can be transduced with the polyclonal TCR-expression lentiviral vectors obtained from a population of T cells (e.g., tumor-infiltrating T cells) some of which are tumor reactive (or tumor specific). The transduced reporter cells can be incubated with tumor cells, tumor tissue, tumor spheres, or APC (either autologous APC or allogeneic APC engineered to express autologous MHC) engineered to express tumor genes (i.e., tumor mRNA-loaded APC which has been studied as cancer vaccine). If the TCR transduced into the reporter cell line is tumor-reactive, the reporter gene or a marker gene in the reporter cell can be expressed at a higher (or in some cases lower) level, and the cell can be identified (e.g., selected/isolated/enriched using FACS or MACS). Optionally, the identified TCRs that are tumor-reactive can be sequenced. The already-fused TCRs from the sorted cells can be simply PCR-amplified and cloned into TCR-expressing vectors in batch. Optionally, individual TCRs can be obtained, for example by picking E. coli colonies hosting the TCR-expressing vector.

In some embodiments, the selected/isolated/enriched reporter-positive or marker-positive cells (e.g., post-selection TCR-programmed recipient cells) are not purely antigen-reactive or tumor-reactive. However, the frequency, or relative abundance of the antigen- or tumor-reactive cells in the selected/isolated/enriched TCR-programmed recipient cells may be nevertheless higher than that in the TCR-programmed recipient cells before the selection (e.g., pre-selection TCR-programmed recipient cells). To facilitate the identification of the antigen- or tumor-reactive TCRs, the TCR repertoire of the post-selection TCR-programmed recipient cells can be sequenced. In some embodiments, the TCR repertoire of the pre-selection TCR-programmed recipient cells can also be sequenced. If the frequency of a TCR in the post-selection TCR-programmed recipient cells is higher than the frequency the TCR in the pre-selection TCR-programmed recipient cells by at least 1.5-fold, 1.8-fold, 2.0-fold, 2.5-fold, 3.0-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10-fold, 15-fold, 20-fold, 30-fold, 40-fold, 50-fold, 60-fold, 70-fold, 80-fold, 90-fold, 100-fold or more, the TCR may be regarded as a putative antigen- or tumor-reactive TCR.

In some embodiments, to further ensure that a TCR is enriched because it recognizes the intended antigen rather than an unintended antigen, a negative-control selection can be performed. The selection where the intended antigen is provided may be called ‘intended selection’. In the negative-control selection, one or more unintended antigen may be provided to bind the TCRs expressed on the TCR-programmed recipient cells. For example, if the intended antigens are a group of mutated antigens (e.g., neoantigens), the unintended antigens can be the wildtype counterparts of the neoantigens. For example, the sequence the minigene for a neoantigen may encode the sequence TEYKLVVVGA[D]GVGKSALTIQLIQN (SEQ ID NO: 6), which is a fragment of the KRAS G12D protein, where the ‘D’ residue in bracket is a mutated residue. The wildtype counterpart of this neoantigen may be TEYKLVVVGA[G]GVGKSALTIQLIQN (SEQ ID NO: 7), where the ‘G’ in bracket is the wildtype residue. The reporter- or marker-positive TCR-programmed recipient cells from the negative-control selection can be selected/isolated/enriched by sorting such as FACS and MACS. If the frequency of a TCR in the selected/isolated/enriched cells from the intended selection is substantially higher than that from the negative-control selection, the TCR may be regarded as a putative antigen-reactive TCR.

A pool of minigenes encoding the neoantigens can be introduced to the APC, which can be mixed with TCR-programmed recipient cells. Reporter- or marker-positive TCR-programmed recipient cells can be selected/isolated/enriched by sorting such as FACS and MACS, resulting in a first population post-selection TCR-programmed recipient cells. In parallel, a negative-control selection may be performed as follows. A pool of minigenes encoding wildtype counterparts of the neoantigens can be introduced to another batch of APC, which can be mixed with TCR-programmed recipient cells. Reporter- or marker-positive TCR-programmed recipient cells can be selected/isolated/enriched by sorting such as FACS and MACS, resulting in a second population post-selection TCR-programmed recipient cells. If the frequency of a TCR in the first post-selection TCR-programmed recipient cells is higher than the frequency the TCR in the second post-selection TCR-programmed recipient cells by at least 1.5-fold, 1.8-fold, 2.0-fold, 2.5-fold, 3.0-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10-fold, 15-fold, 20-fold, 30-fold, 40-fold, 50-fold, 60-fold, 70-fold, 80-fold, 90-fold, 100-fold or more, the TCR may be regarded as a putative neoantigen-reactive TCR.

As another example, amplified total mRNA from a cancer tissue (e.g., colon cancer tissue) can be introduced to one batch of APC to be used for the intended selection. In parallel, amplified total mRNA from a healthy tissue of the same organ (e.g., healthy colon tissue) can be introduced to another batch of APC to be used for the negative-control selection.

To sequence the TCR repertoire and analyze the frequency of each TCR in a population of cells, the CDR3 sequences of the beta chain or the CDR3 sequences of the alpha chain or both can be analyzed. Nucleic acid molecules (e.g., DNA or mRNA) encoding the CDR3 sequences can be amplified for analyzing the TCR repertoire.

For example, the method provided herein can comprise providing a plurality of T cells expressing a plurality of TCRs, where each T cell of the plurality of T cells can expresse a cognate pair of a TCR of the plurality of TCRs. Next, a first polynucleotide encoding a first TCR chain and a second polynucleotide encoding a second TCR chain of the cognate pair of the TCR of each T cell can be paired to generate a plurality of polynucleotide pairs. Polynucleotides comprising the plurality of polynucleotide pairs can be delivered into a first plurality of recipient cells and a second plurality of recipient cells, where each recipient cell of the first or the second plurality of recipient cells can comprise a polynucleotide comprising at least one polynucleotide pair of the plurality of polynucleotide pairs. The plurality of polynucleotide pairs can be expressed in the first and second plurality of recipient cells. The first plurality of recipient cells can be contacted with a first antigen, thereby activating a first marker of a first subset of the first plurality of recipient cells. The second plurality of recipient cells with a second antigen, thereby activating a second marker of a second subset of the second plurality of recipient cells. The second antigen may not be homologous to the first antigen. The first antigen and the second antigen may be derived from the same protein. The first antigen may comprise a mutated sequence and the second antigen may comprise a wildtype sequence. The first antigen may be derived from a cancer cell and the second antigen may be derived from a healthy cell. For example, the first antigen may be a neoantigen, and the second antigen may be a wildtype antigen. Next, the first subset can be isolated based on the first marker and the second subset can be isolated based on the second marker. Next, a TCR repertoire of the first subset of the first plurality of recipient cells and a TCR repertoire of the second subset of the second plurality of recipient cells can be sequenced. A frequency of a TCR of the TCR repertoire in the first subset of the first plurality of recipient cells and a frequency of a TCR of the TCR repertoire in the second subset of the second plurality of recipient cells can be determined. The TCR having a frequency in the first subset of the first plurality of recipient cells that is higher than its frequency in the second subset of the second plurality of recipient cells can be identified as the putative target-reactive TCR. Optionally, the method can further comprise, prior to contacting, sequencing a TCR repertoire of the plurality of recipient cells, and determining a frequency of a TCR of the TCR repertoire in the plurality of recipient cells.

The methods described herein can enable the identification of tumor-reactive TCRs for personalized TCR-T therapy or an immuno-monitoring test to quantify tumor-reactive T cells in tumor tissue or peripheral blood of a patient. For example, a tumor biopsy can be obtained from a cancer patient. Peripheral or tumor-infiltrating T cells can also be obtained from the same patient. T cells expressing a certain marker (e.g., PD1, CD137, CD39, CD69, Tim3) or the combination thereof can be selected/isolated/enriched using existing methods. The TCRs from the peripheral T cells or a subpopulation thereof can be cloned into TCR-expressing vectors, for example, using single-cell reactor-based methods described herein. The informatically paired TCRs sequences from a population of T cells with a particular transcriptome signature (e.g., naïve-like, exhausted, memory, stem cell memory, or central memory TCF7+) can be obtained using sequencing (e.g., single cell RNA-Seq). The paired TCR-encoding polynucleotide and TCR-expressing vectors can be synthesized using gene assembly methods such as the TCR gene self-assembly methods described herein.

These TCRs can in turn be used to engineer TCR-engineered reporter cell lines (e.g., recipient cells) as described herein. Meanwhile, the HLA genes can be amplified from peripheral blood. An APC cell line with no human HLA expression (e.g., a human cell line expressing no or very low level of MHC such as K562 and 721.221, a non-human primate cell line such as COS-7 or a human cell line with endogenous HLA knocked out) can be engineered to express the HLA genes of the patient. The autologous APC (e.g., monocyte-derived dendritic cells, dendritic cells, macrophages, and B cells) from the patient may also be used as APC. The full-length mRNA from the tumor sample (e.g., surgical sample or biopsy) can be isolated, amplified and transfected to the autologous or HLA-engineered allogeneic APC described above to create tumor mRNA-loaded APC. The tumor sample can be a biopsy sample such as core biopsy or fine needle biopsy sample. These sample may have a small volume (e.g., <1000 mm³, <500 mm³, <100 mm³, <50 mm³) because even a small volume of tumor sample may contain sufficient mRNA to be amplified. In some cases, the volume of a tumor sample can be equal to or at most about 2000 mm³, 1000 mm³, 800 mm³, 500 mm³, 100 mm³, 50 mm³, or 20 mm³. Thus, this method can be applicable to situations where large surgical tumor sample is difficult to obtain. The TCR-engineered reporter cells and the tumor mRNA-loaded APC can be co-incubated and the reporter-expressing cells, which are tumor-reactive, can be isolated as described above. The TCRs from the isolated cells can be sequenced to provide the sequences and abundance of tumor-reactive TCRs. A report containing such information can be issued. This method can be combined with conventional TCR repertoire analysis to improve the accuracy of the abundance of tumor-reactive TCRs. The methods to obtain an engineered APCs and tumor mRNA-loaded APCs described in this paragraph can also be used in methods described elsewhere in the present disclosure.

For example, a method of identifying a plurality of target-reactive T-cell receptors (TCRs) can comprise providing a population of cells expressing a population of TCRs. The population of TCRs may be exogenous to the population of cells. The population of TCRs may comprise different cognate pairs, for example, at least about 5, 10, 15, 20, 25, 30, 35, 40, 45, 50, 55, 60, 65, 70, 75, 80, 85, 90, 95, 100, 500, 1,000, 10,000, 1,000,000 or more different cognate pairs. The population of TCRs may comprise V regions from at least about 2, 5, 10, 15, 20, 25, 30, 35, 40, or more different V genes. The population of TCRs may comprise at least 100 different VJ combinations. The method can further comprise contacting the population of cells with one or more target antigens, wherein the plurality of target-reactive TCRs bind to the one or more target antigens. The plurality of target-reactive TCRs can then be isolated or enriched. In some cases, the plurality of at least about 5, 10, 15, 20, 30, 50, 100, 200, 300, 400, 500, 600, or more target-reactive TCRs can be isolated or enriched. The population of cells can be engineered cells, non-exhausted cells, or cells not isolated from a patient. The method can further comprise contacting the population of cells with one or more target antigens, wherein the plurality of target-reactive TCRs can bind to the one or more target antigens. The plurality of at least 5 target-reactive TCRs can then be isolated or enriched. The population of TCRs can comprise at least about 100, 200, 500, 1,000, 10,000, 100,000, 1,000,000, or 10,000,000 different cognate pairs. The plurality of target-reactive TCRs comprises V regions from at least 10, at least 15, at least 20, or more different V genes. In some cases, the population of cells is contacted with tumor cells or antigen-presenting cells presenting the one or more target antigens. The target antigens can be tumor antigens or tissue-specific antigens. The one or more target antigens can be in complex with a major histocompatibility complex (MHC). The MHC can be an MHC tetramer. The method can further comprise administering at least one target-reactive TCR of the plurality of target-reactive TCRs into a subject. In some cases, a cell of the population of cells or engineered cells can comprise a reporter gene. The reporter gene can be regulated to send a signal when a TCR of the cell binds to a target antigen of the one or more target antigens. The population of cells or engineered cells can be cell line cells (e.g., Jurkat cells).

For example, a method of identifying a plurality of target-reactive T-cell receptors (TCRs) can comprise providing a plurality of T cells expressing a plurality of TCRs. The plurality of TCRs may comprise at least 5, 10, 15, 20, 25, 30, 35, 40, 45, 50, 55, 60, 65, 70, 75, 80, 85, 90, 95, 100, 500, 1,000, 10,000, 1,000,000 or more different cognate pairs comprising V regions from at least about 2, 5, 10, 15, 20, 25, 30, 35, or more different V genes. The method can further comprise physically linking a first polynucleotide encoding a TCR alpha (or gamma) chain and a second polynucleotide encoding a TCR beta (or delta) chain of each TCR of the plurality of TCRs, thereby generating a plurality of fused polynucleotides. The plurality of fused polynucleotides can be expressed in a plurality of cells, wherein a subset of the plurality of cells expresses the plurality of target-reactive TCRs. The plurality of cells can be contacted with one or more target antigens to identify the plurality of target-reactive TCRs. The subset of the plurality of cells expressing the plurality of target-reactive TCRs can bind to the one or more target antigens. The subset of the plurality of cells can be isolated or enriched, thereby isolating or enriching the plurality of target-reactive TCRs.

For example, a method of identifying a plurality of target-reactive T-cell receptors (TCRs) can comprise providing a plurality of T cells expressing a plurality of TCRs. The plurality of TCRs may comprise at least 5, 10, 15, 20, 25, 30, 35, 40, 45, 50, 55, 60, 65, 70, 75, 80, 85, 90, 95, 100, 500, 1,000, 10,000, 1,000,000 or more different cognate pairs comprising V regions from at least 2, 5, 10, 15, 20, 25, 30, 35, or more different V genes. The method can further comprise sequencing one or more cognate pairs of the plurality of TCRs without using any barcoding, e.g., single cell barcoding. For example, sequencing can comprise sequencing TCR chains of the one or more cognate pairs of the plurality of TCRs, wherein the TCR chains do not comprise a same barcode. The one or more cognate pairs encoding the plurality of TCRs or a subset thereof can then be expressed, for example, in soluble form or in a plurality of cells. The plurality of cells used to express the one or more cognate pairs can be cell line cells. The plurality of TCRs or the subset thereof may comprise the plurality of target-reactive TCRs. The plurality of TCRs can then be contacted with one or more target antigens to identify target-reactive TCRs. The plurality of target-reactive TCRs can bind to the one or more target antigens and can then be isolated or enriched. In some cases, when identifying cognate pairs of the TCRs, a first polynucleotide encoding a TCR alpha chain and a second polynucleotide encoding a TCR beta chain of each TCR of the plurality of TCRs can be physically linked, thereby generating a plurality of fused polynucleotides. The method may further comprise sequencing the one or more cognate pairs of the plurality of TCRs. The plurality of T cells can be isolated from a subject. The plurality of T cells can be tumor-infiltrating T cells. The plurality of T cells can comprise exhausted T cells. The plurality of target-reactive TCRs can be isolated or enriched by FACS. The plurality of target-reactive TCRs can be isolated by a cell surface marker or a cytokine marker. For example, the target-reactive TCRs can be isolated or enriched by using antibodies specific to a surface marker such as CD69, CD25 or 41BB for sorting by FACS.

In some cases, a plurality of T cells isolated from a sample can be cultured and stimulated in vitro, for example, with APCs presenting antigens, and a subset of the plurality of T cells can be enriched. These pre-enriched T cells can then be used to identify the target-reactive TCRs. For example, when isolating a plurality of T cells from a blood sample or a PBMC sample, a small fraction of the plurality of T cells may be target-reactive T cells. In such cases, the plurality of T cells can be contacted with one or more target antigens (e.g., in MHC tetramer form or presented on a cell surface) first to activate the T cells. A subset of the plurality of T cells can be enriched or isolated based on a marker (e.g., a surface marker), which can then be used for the subsequent identification methods described herein including fusing the cognate TCR chains. The pre-enriched T cells may also be subject to other methods to identify cognate pairs, for example, using sequencing. The sequencing may use single cell barcoding (e.g., partitioning T cells into individual compartment, barcoding nucleic acids released from a single cell, sequencing the nucleic acids and pair the TCR chains from a single cell based on a same barcode).

In some cases, the method provided herein can comprise providing a plurality of cells expressing a plurality of TCRs, each cell of the plurality of cells expressing a TCR of the plurality of TCRs. The plurality of TCRs can comprise at least 5, 10, 20, 50 or more different cognate pairs. In some cases, the plurality of TCRs can further comprise V regions from a plurality of V genes. The plurality of TCRs can be exogenous to the plurality of cells. Optionally, the TCR repertoire of the plurality of cells can be sequenced, and a frequency of a TCR of the TCR repertoire in the plurality of cells can be determined. The plurality of cells can be contacted with one or more antigens, thereby activating a marker of a subset of the plurality of cells. The subset of the plurality of cells can be isolated based on the marker. Next, subsequent to isolation, the TCR repertoire of the subset of the plurality of cells can be sequenced, and a frequency of a TCR of the TCR repertoire in the subset of the plurality of cells can be determined. The TCR having a frequency in the subset of the plurality of cells that is higher than its frequency in the plurality of cells can be identified as the putative target-reactive TCR.

In some cases, the method provided herein can comprise providing a first plurality of cells expressing a plurality of TCRs and a second plurality of cells expressing the plurality of TCRs, each cell of the first or the second plurality of cells expressing a TCR of the plurality of TCRs. The first plurality of cells and the second plurality of cells can be from a same sample. For example, the first plurality of cells and the second plurality of cells can be two aliquots of a sample. The plurality of TCRs can comprise at least 5, 10, 20, 50 or more different cognate pairs. In some cases, the plurality of TCRs can further comprise V regions from a plurality of V genes. The plurality of TCRs are exogenous to the first or the second plurality of cells. Next, the first plurality of cells can be contacted with a first antigen, thereby activating a first marker of a first subset of the first plurality of cells, and the second plurality of cells can be contacted with a second antigen, thereby activating a second marker of a second subset of the second plurality of cells. The first subset can be isolated based on the first marker and the second subset can be isolated based on the second marker. The TCR repertoire of the first subset of the first plurality of cells and the TCR repertoire of the second subset of the second plurality of cells can be sequenced. The frequency of a TCR of the TCR repertoire in the first subset of the first plurality of cells and the frequency of a TCR of the TCR repertoire in the second subset of the second plurality of cells can be determined. The TCR having a frequency in the first subset of the first plurality of cells that is higher than its frequency in the second subset of the second plurality of cells can be identified as the putative target-reactive TCR. The frequency can be determined by sequencing reads of a particular TCR divided by total sequencing reads of the TCR repertoire. In some cases, the frequency can be determined by unique molecule index (UMI) count of a particular TCR divided by total UMI count of the TCR repertoire. The UMI can be added to the nucleic acid molecules to be sequenced when preparing the sequencing library using standard protocols.

The identified putative target-reactive TCR described herein can be isolated from the pre-selection or post-selection pool of TCR-programmed recipient cells. In some cases, the identified putative target-reactive TCR can be synthesized (e.g., chemically synthesized). In some cases, the identified putative target-reactive TCR can be selected (e.g., amplified or dialed out) from pre-selection or post-selection pool using nucleic acid amplification methods such as dial-out PCR. For example, in various cases described herein, the polynucleotide encoding a TCR (e.g., a polynucleotide of the expressible TCR polynucleotide library) can comprise a barcode. The barcode can be a unique barcode to a particular TCR sequence within the pool. The sequences of the barcode can be arbitrarily designed. The sequences of the barcode can be designed to avoid common pitfalls such as unwanted secondary structures, restriction sites, similarity with other sequences in the TCR genes, or similarities between primer-binding sites. The barcode can be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more nucleotides long. The barcode can be an additional sequence added to the paired TCR sequence, or a sequence already included in each sequence of the paired TCR pool. For example, a connector sequence or portion thereof can be used as a barcode when generating paired TCR using self-assembly described herein. For another example, a barcode sequence can be introduced in the vector when preparing the expressible TCR polynucleotide library. The identified putative target-reactive TCRs can then be amplified using a primer pair targeting the barcode sequence and a common sequence.

### Methods of Pairing TCRs

The polyclonal TCRs obtained from source TCR-expressing cells can be paired physically or informatically. Examples of methods used to pair TCRs are provided herein.

### Single-Cell Reactor-Based Methods

The TCRs from a source TCR-expressing cell can be paired (e.g., physically fused) within a single-cell reactor. A single-cell reactor can be a compartment in which molecules of interest from a single biological particle (e.g., a single cell) can react with a reagent or with each other. A single-cell reactor may comprise two components: (1) a solid support to which molecules of interest from a single cell can be associated, and (2) an aqueous content for the biochemical reactions to happen. Molecules of interest in single-cell reactors may undergo reactions during which molecules of interest from different cells do not contact each other or mix. The molecules of interest may be nucleic acids, proteins or other molecules present in a cell. The nucleic acids may be DNA, RNA, mRNA, miRNA, tRNA, etc. The nucleic acids may encode an immunoreceptor or an immunoreceptor chain. The single-cell reactor can be a compartment or a vessel. The compartment or the vessel can be a droplet, a tube, a well, a discrete region on an array, or a hardened particle (e.g., a hydrogel particle).

In an aspect, a method for preparing a fused bipartite immunoreceptor polynucleotide library, can comprise: (a) generating a plurality of vessels, each comprising (1) a cell, wherein the cell comprises a first nucleic acid encoding a first peptide chain of a bipartite immunoreceptor and a second nucleic acid encoding a second peptide chain of the bipartite immunoreceptor, and (2) a plurality of polymerizable or gellable polymers and/or monomers; and (b) polymerizing or gelling the plurality of polymerizable or gellable polymers and/or monomers to form a plurality of hardened particles, each hardened particle of the plurality having a matrix composed of the polymerized or gelled plurality of polymers and/or monomers, wherein each hardened particle of the plurality comprises a first primer extension product of the first nucleic acid and a second primer extension product of the second nucleic acid; wherein the first primer extension product and the second primer extension product are embedded or entrapped within the matrix, and wherein diffusion of the first primer extension product and the second primer extension product are restricted.

The first and the second primer extension product can be a reverse transcription (RT) product, a second strand synthesis (SSS) product, or an amplification product. The first and/or the second primer extension product can comprise an adaptor sequence. In some embodiments, the adaptor sequence is not hybridizable or complementary to the first or the second nucleic acid molecule. In some embodiments, the first and the second primer extension product encode a variable domain. In some embodiments, the variable domain comprises CDR1, CDR2, and CDR3. In some embodiments, the first and/or the second primer extension product further encodes a constant domain.

In some embodiments, the method further comprises lysing the cell to release the first nucleic acid and the second nucleic acid. In some embodiments, the method further comprises reverse transcribing the first nucleic acid and the second nucleic acid. In some embodiments, the reverse transcribing is performed by using a RT primer. In some embodiments, the RT primer is linked to a diffusion-restricting agent, wherein the diffusion-restricting agent restricts diffusion of the RT primer within the matrix. In some embodiments, the method further comprises performing a template-switch reaction or a SSS reaction. In some embodiments, the method further comprises amplifying the first nucleic acid and the second nucleic acid to generate a first and a second amplification product. In some embodiments, for each of the first or the second nucleic acid, the amplifying is performed by using a first amplification primer and a second amplification primer. In some embodiments, the first amplification primer is linked to a diffusion-restricting agent, wherein the diffusion-restricting agent restricts diffusion of the first amplification primer within the matrix.

In some embodiments, the method further comprises washing the plurality of hardened particles. In some embodiments, the method further comprises washing the plurality of hardened particles to allow a reagent to diffuse out from the plurality of hardened particles. In some embodiments, the reagent comprises a RT primer, an amplification primer, a template-switch primer, a SSS primer, or any combination thereof. In some embodiments, the method further comprises repeatedly washing the plurality of hardened particles. In some embodiments, the method further comprises emulsifying the plurality of hardened particles in oil after a washing step, thereby forming an additional plurality of vessels, each vessel of the additional plurality of vessels comprising a single hardened particle of the plurality of hardened particles. In some embodiments, the first and the second primer extension product are linked to a diffusion-restricting agent. In some embodiments, the diffusion-restricting agent is a polymer. In some embodiments, the polymer is a polyacrylamide, a polyethylene glycol, or a polysaccharide. In some embodiments, the diffusion restricting agent is a particle. In some embodiments, the particle has a diameter that is larger than a pore size of the matrix. In some embodiments, the diffusion restricting agent is the matrix. In some embodiments, the first and the second primer extension product is linked to the diffusion-restricting agent through a capture agent. In some embodiments, the capture agent comprises an immobilization moiety. In some embodiments, the immobilization moiety links the capture agent to the diffusion-restricting agent.

In some embodiments, the immobilization moiety comprises a reactive group. In some embodiments, the capture agent further comprises a targeting moiety. In some embodiments, the targeting moiety is a capture oligonucleotide. In some embodiments, the first amplification primer comprises an oligonucleotide sequence that hybridizes to the capture oligonucleotide. In some embodiments, the first and the second amplification product comprise the oligonucleotide sequence that hybridizes to the capture oligonucleotide, thereby linking the first and the second amplification product to the capture agent and thereby linking to the diffusion-restricting agent. In some embodiments, the reactive group is a succinimidyl ester, an amide, an acrylamide, an acyl azide, an acyl halide, an acyl nitrile, an aldehyde, a ketone, an alkyl halide, an alkyl sulfonate, an anhydride, an aryl halide, an aziridine, a boronate, a carbodiimide, a diazoalkane, an epoxide, a haloacetamide, a haloplatinate, a halotriazine, an imido ester, an isocyanate, an isothiocyanate, a maleimide, a phosphoramidite, a silyl halide, a sulfonate ester, a sulfonyl halide, an amine, an aniline, a thiol, an alcohol, a phenol, a hyrazine, a hydroxylamine, a carboxylic acid, a glycol, or a heterocycle. In some embodiments, the method further comprises linking the first amplification product and the second amplification product to form a fused bipartite immunoreceptor polynucleotide within each vessel of the additional plurality of vessels, thereby generating the fused bipartite immunoreceptor polynucleotide library having a plurality of fused bipartite immunoreceptor polynucleotides. In some embodiments, the first amplification product and the second amplification product are linked by ligation or PCR. In some embodiments, the first amplification product and the second amplification product are linked by a phosphodiester bond to form a continuous polynucleotide. In some embodiments, the first amplification product and the second amplification product are linked in-frame.

In some embodiments, the method further comprises releasing the plurality of fused bipartite immunoreceptor polynucleotides from the additional plurality of vessels. In some embodiments, the method further comprises circularizing each fused bipartite immunoreceptor polynucleotide of the plurality. In some embodiments, the method further comprises inserting each fused bipartite immunoreceptor polynucleotide of the plurality into a vector. In some embodiments, the vector is a self-amplifying RNA replicon, a plasmid, a phage, a transposon, a cosmid, a virus, or a virion. In some embodiments, the vector is a viral vector. In some embodiments, the viral vector is derived from a retrovirus, a lentivirus, an adenovirus, an adeno-associated virus, a herpes virus, a pox virus, an alpha virus, a vaccina virus, a hepatitis B virus, a human papillomavirus or a pseudotype thereof. In some embodiments, the vector is a non-viral vector. In some embodiments, the non-viral vector is a nanoparticle, a cationic lipid, a cationic polymer, a metallic nanopolymer, a nanorod, a liposome, a micelle, a microbubble, a cell-penetrating peptide, or a liposphere. In some embodiments, the bipartite immunoreceptor is a T-cell receptor (TCR). In some embodiments, the TCR comprises a TCR alpha peptide chain and a TCR beta peptide chain, or a TCR gamma peptide chain and a TCR delta peptide chain. In some embodiments, the cell is an immune cell. In some embodiments, the immune cell is a lymphocyte. In some embodiments, the lymphocyte is a T cell. In some embodiments, the T cell is an inflammatory T cell, a cytotoxic T cell, a regulatory T cell, a helper T cell, a natural killer T cell, or a combination thereof. In some embodiments, the T cell is a CD4+ T cell or a CD8+ T cell. In some embodiments, the immune cell is isolated from a tumor tissue or a blood sample. In some embodiments, the method further comprises delivering the fused bipartite immunoreceptor polynucleotide into a host cell. In some embodiments, the fused bipartite immunoreceptor polynucleotide library comprise at least 50, at least 100, at least 200, at least 500, at least 1,000, at least 10,000, at least 100,000, at least 1,000,000, or at least 10,000,000 different fused bipartite immunoreceptor sequences. In some embodiments, the first peptide chain and the second peptide chain are a cognate pair of the bipartite immunoreceptor. In some embodiments, the vessel is a droplet. In some embodiments, the droplet is a water-in-oil droplet. In some embodiments, the hardened particle is a hydrogel particle. In some embodiments, the polymers are polysaccharides, polyacrylamides, or a combination thereof. In some embodiments, the polysaccharides are agarose, hyaluronic acids, carboxymethycellose, chitosan, or alginate. In some embodiments, the monomers are acrylamide or methacrylamide monomers. In some embodiments, the polymerized or gelled plurality of polymers and/or monomers comprises a mixture of agarose and polyacrylamides. In some embodiments, the polymerized or gelled plurality of polymers and/or monomers is cross-linked. In some embodiments, polymerizing or gelling the plurality of polymerizable or gellable polymers and/or monomers comprises using an initiator. In some embodiments, the initiator is a UV light or a chemical. In some embodiments, polymerizing or gelling the plurality of polymerizable or gellable polymers and/or monomers comprises reducing temperature of the vessel.

The methods provided herein can be performed in a liquid, comprising: (a) extending a first oligonucleotide hybridized to a nucleic acid molecule, thereby forming a first extension product; (b) amplifying the first extension product or a reverse complement strand thereof with a primer set comprising a first primer and a second primer, thereby forming a first amplification product; (c) generating a polymer matrix in the liquid to form a hydrogel particle, thereby restricting diffusion of the first amplification product; and (d) washing the hydrogel particle, thereby depleting the second primer from the hydrogel particle. In some embodiments, the first primer or the first amplification product is linked to a diffusion-restricting agent.

In some cases, a method performed in a liquid can comprise: (a) extending a first oligonucleotide hybridized to a nucleic acid molecule, thereby forming a first extension product; (b) generating a polymer matrix in the liquid to form a hydrogel particle, thereby restricting diffusion of the first extension product or a reverse complement strand thereof; (c) washing the hydrogel particle; and (d) amplifying the first extension product or the reverse complement strand thereof with a primer set comprising a first primer and a second primer, thereby forming a first amplification product.

In some embodiments, the first oligonucleotide or the first extension product is linked to a diffusion-restricting agent. In some embodiments, the method further comprises extending a second oligonucleotide hybridized to an additional nuclei acid molecule. In some embodiments, the nucleic acid molecule and the additional nucleic acid molecule encode a first peptide chain and a second peptide chain of an immunoreceptor, wherein the first peptide chain and the second peptide chain are a cognate pair of the immunoreceptor. In some embodiments, the diffusion-restricting agent is a polymer or a particle. In some embodiments, the polymer is a polyacrylamide, a polyethylene glycol, or a polysaccharide. In some embodiments, the particle has a diameter that is larger than a pore size of the polymer matrix. In some embodiments, the diffusion-restricting agent is the polymer matrix. In some embodiments, the nucleic acid molecule is DNA or RNA. In some embodiments, the nucleic acid molecule is a genomic DNA. In some embodiments, the nucleic acid molecule is a messenger RNA. In some embodiments, the first oligonucleotide is a reverse transcription (RT) primer. In some embodiments, the method further comprises extending the RT primer with a template-switch oligonucleotide, thereby generating the first extension product having a reverse complement sequence of the template-switch oligonucleotide. In some embodiments, the method further comprises using a second strand synthesis (SSS) primer having an adaptor sequence to synthesize the reverse complement strand of the first extension product. In some embodiments, the adaptor sequence is not hybridizable or complementary to the nucleic acid molecule or the first extension product. In some embodiments, the first extension product comprises the adaptor sequence. In some embodiments, the nucleic acid molecule encodes a peptide chain of an immunoreceptor. In some embodiments, the method further comprises, after or during washing the hydrogel particle, contacting a reagent with the hydrogel particle such that the reagent diffuses into the hydrogel particle. In some embodiments, the reagent is an oligonucleotide or an enzyme. In some embodiments, the enzyme is a polymerase. In some embodiments, the method further comprises emulsifying the hydrogel particle in oil after washing.

In some cases, a method performed in a liquid can comprise: (a) forming a plurality of droplets, wherein at least two droplets of the plurality comprise a single cell; (b) extending a first oligonucleotide hybridized to a first nucleic acid molecule from the single cell, thereby forming a first extension product; and extending a second oligonucleotide hybridized to a second nucleic acid molecule from the single cell, thereby forming a second extension product; (c) amplifying the first extension product or a reverse complement strand thereof with a first primer set comprising a first primer and a second primer, thereby forming a first set of amplification products; and amplifying the second extension product or a reverse complement strand thereof with a second primer set comprising a third primer and a fourth primer, thereby forming a second set of amplification products; and (d) linking an amplification product of the first set of amplification products to an amplification product of the second set of amplification products, wherein linking comprises linking in the liquid in the absence of the second and the fourth primer.

In some cases, a method performed in a liquid can comprise: (a) forming a plurality of droplets, wherein at least two droplets of the plurality comprise a single cell; (b) extending a first oligonucleotide hybridized to a first nucleic acid molecule from the single cell, thereby forming a first extension product; and extending a second oligonucleotide hybridized to a second nucleic acid molecule from the single cell, thereby forming a second extension product; (c) amplifying the first extension product or a reverse complement strand thereof with a first primer set comprising a first primer and a second primer, thereby forming a first set of amplification products; and amplifying the second extension product or a reverse complement strand thereof with a second primer set comprising a third primer and a fourth primer, thereby forming a second set of amplification products; (d) removing the second and the fourth primer; and linking an amplification product of the first set of amplification products to an amplification product of the second set of amplification products.

In some embodiments, each droplet comprises a plurality of polymerizable or gellable polymers and/or monomers. In some embodiments, the method further comprises generating a polymer matrix in the liquid to form a hydrogel particle, thereby restricting diffusion of the first set of amplification products and the second set of amplification products. In some embodiments, the method further comprises washing the hydrogel particle, thereby depleting the second primer and the fourth primer from the hydrogel particle. In some embodiments, linking comprises generating a sticky end on the amplification product of the first and the second set. In some embodiments, generating the sticky end on the amplification product comprises using a USER enzyme. In some embodiments, linking comprises hybridizing the amplification product of the first and the second set. In some embodiments, linking comprises ligating the amplification product of the first and the second set. In some embodiments, the first primer and the third primer are the same primer. In some embodiments, the first primer, the third primer, the first set of amplification products, or the second set of amplification products is linked to a diffusion-restricting agent.

In some cases, a method performed in a liquid can comprise: (a) forming a plurality of droplets, wherein at least two droplets of the plurality comprise a single cell; (b) extending a first oligonucleotide hybridized to a first nucleic acid molecule from the single cell, thereby forming a first extension product; and extending a second oligonucleotide hybridized to a second nucleic acid molecule from the single cell, thereby forming a second extension product; (c) generating a polymer matrix in the liquid to form a hydrogel particle, thereby restricting the diffusion of the first extension product and the second extension product are restricted; (d) amplifying the first extension product or a reverse complement strand thereof with a first primer set comprising a first primer and a second primer, thereby forming a first set of amplification products; and amplifying the second extension product or a reverse complement strand thereof with a second primer set comprising a third primer and a fourth primer, thereby forming a second set of amplification products; and (e) linking an amplification product of the first set of amplification products to an amplification product of the second set of amplification products.

In some embodiments, the method further comprises washing the hydrogel particle after (c). In some embodiments, the method further comprises contacting a reagent with the hydrogel particle such that the reagent diffuses into the hydrogel particle. In some embodiments, the reagent comprises an enzyme or an oligonucleotide. In some embodiments, the oligonucleotide comprises the first primer set and/or the second primer set. In some embodiments, the enzyme is a polymerase, a ligase, a USER enzyme, or a combination thereof. In some embodiments, the method further comprises emulsifying the hydrogel particle in oil after washing. In some embodiments, the first oligonucleotide or the second oligonucleotide is linked to a diffusion-restricting agent. In some embodiments, the first oligonucleotide or the second oligonucleotide is a RT primer. In some embodiments, the method further comprises using a second strand synthesis (SSS) primer to synthesize the reverse complement strand of the first and/or the second extension product. In some embodiments, the SSS primer comprises an adaptor sequence. In some embodiments, the adaptor sequence is not hybridizable or complementary with the first and/or the second extension product. In some embodiments, the method further comprises extending the RT primer with a template-switch oligonucleotide. In some embodiments, the single cell is an immune cell. In some embodiments, the immune cell is a T cell or a B cell. In some embodiments, the first nucleic acid molecule and the second nucleic acid molecule are DNA or RNA. In some embodiments, the DNA is a genomic DNA. In some embodiments, the RNA is a messenger RNA. In some embodiments, the first nucleic acid molecule encodes a first peptide chain of an immunoreceptor and the second nucleic acid molecule encodes a second peptide chain of the immunoreceptor. In some embodiments, the first peptide chain and the second peptide chain are a cognate pair of the immunoreceptor. In some embodiments, the first peptide chain or the second peptide chain comprises a variable domain. In some embodiments, the variable domain comprises a CDR1, CDR2, CDR3, or a combination thereof. In some embodiments, the first peptide chain or the second peptide chain comprises a constant domain. In some embodiments, the first peptide chain or the second peptide chain comprises a transmembrane region and/or a cytoplasmic tail. In some embodiments, the immunoreceptor is a B-cell receptor. In some embodiments, the immunoreceptor is a T-cell receptor. In some embodiments, the diffusion-restricting agent is a polymer or a particle. In some embodiments, the polymer is a polyacrylamide, a polysaccharide, or a polyethylene glycol. In some embodiments, the particle has a diameter that is larger than a pore size of the hydrogel particle. In some embodiments, the diffusion-restricting agent is the polymer matrix. In some embodiments, linking comprises generating a sticky end on the amplification product of the first and the second set. In some embodiments, generating the sticky end on the amplification product comprises using a USER enzyme. In some embodiments, linking comprises hybridizing the amplification product of the first and the second set. In some embodiments, linking comprises ligating the amplification product of the first and the second set.

The methods provided herein can comprise: (1) providing a plurality of at least 1,000 cells, each cell of the at least 1,000 cells comprising a TCR alpha chain and a TCR beta chain; (2) providing a plurality of at least 1,000 compartments, each compartment of the at least 1,000 compartments comprising a solid support, wherein the solid support comprises: (a) a first polynucleotide, comprising a first common sequence, a second common sequence, and a protein-coding sequence encoding a TCR alpha chain between the first and the second common sequence, (b) a second polynucleotide, comprising a third common sequence, a fourth common sequence, and a protein-coding sequence encoding a TCR beta chain between the third and the fourth common sequence, wherein, the TCR alpha chain and the TCR beta chain in each compartment is a cognate pair present in at least one of the plurality of cells, thereby providing a first plurality of protein-coding sequences each encoding a TCR alpha chain and a second plurality of protein-coding sequences each encoding a TCR beta chain; and (3) physically linking the first polynucleotide and the second polynucleotide in each compartment. In some embodiments, the first plurality of protein-coding sequences comprises at least 10 TRAV subgroups and the second plurality of protein-coding sequences comprises at least 10 TRBV subgroups. In some embodiments, each compartment of the at least 1,000 compartments comprise a cell from the plurality of at least 1,000 cells. In some embodiments, the compartment is a well, a microwell, or a droplet. In some embodiments, the solid support is a bead, a hydrogel particle, or a surface of the well or microwell. In some embodiments, the first common sequence, the second common sequence, the third common sequence, or the fourth common sequence is the same in the plurality of at least 1,000 compartments.

Additional details for methods for physically linking the TCR chains can be found in International Application PCT/US2019/046170, which is entirely incorporated herein by reference.

### TCR Gene Self-Assembly

The TCRs from source TCR-expressing cells can be paired informatically, for example, using sequencing. Sequences encoding natively paired (or cognate) TCRs can be identified using various methods, including but not limited to using single cell barcoding and sequencing technologies. After obtaining the sequences encoding natively paired TCRs, compositions and methods described herein can be used to construct or assemble one or more nucleic acid sequences to express the natively paired TCRs in any given host cell(s) in a quick, high-throughput and cost-effective manner. The one or more nucleic acid sequences can comprise greater than or equal to about 1, 5, 10, 20, 50, 100, 200, 300, 400, 500, 1,000, 1,500, 2,000, 2,500, 3,000, 3,500, 4,000, 5,000, 6,000, 7,000, 8,000, 9,000, 10,000, 12,000, 15,000, 20,000, 100,000, 1,000,000, 10,000,000, or more different sequences encoding different TCRs.

In an aspect, a method for generating a plurality of nucleic acid molecules can comprise: providing a first plurality of nucleic acid molecules, wherein a nucleic acid molecule of the first plurality of nucleic acid molecules comprises a sequence encoding a first CDR3 of a first T-cell receptor (TCR) chain and a second CDR3 of a second TCR chain, wherein the first CDR3 and the second CDR3 are from a cognate pair of TCR chains; providing a second plurality of nucleic acid molecules, wherein a nucleic acid molecule of the second plurality of nucleic acid molecules comprises a sequence derived from a TCR V gene, wherein the nucleic acid molecule does not comprise a sequence encoding a constant domain; and contacting the first plurality of nucleic acid molecules and the second plurality of nucleic acid molecules, wherein the nucleic acid molecule of the first plurality of nucleic acid molecules links with the nucleic acid molecule of the second plurality of nucleic acid molecules to form a nucleic acid molecule comprising the sequence encoding the first CDR3 and the second CDR3 and the sequence derived from the TCR V gene, wherein the sequence encoding the first CDR3 and the second CDR3 and the TCR V gene are derived from the cognate pair of TCR chains.

In some embodiments, each nucleic acid molecule of the first plurality of nucleic acid molecules comprises a sequence encoding a different first CDR3 of a first TCR chain and/or a different CDR3 of a second TCR chain. In some embodiments, each nucleic acid molecule of the second plurality of nucleic acid molecules comprises a sequence derived from a different TCR V gene. In some embodiments, the first plurality of nucleic acid molecules and the second plurality of nucleic acid molecules are contacted in a same compartment. In some embodiments, the nucleic acid molecule of the first plurality of nucleic acid molecules further comprises a connector sequence, wherein the connector sequence links the nucleic acid molecule of the first plurality of nucleic acid molecules and the nucleic acid molecule of the second plurality of nucleic acid molecules. In some embodiments, the nucleic acid molecule of the second plurality of nucleic acid molecules further comprises an anti-connector sequence, which anti-connector sequence is complementary to the connector sequence. In some embodiments, the connector sequence hybridizes to the anti-connector sequence to link the nucleic acid molecule of the first plurality of nucleic acid molecules and the nucleic acid molecule of the second plurality of nucleic acid molecules. In some embodiments, the connector sequence is codon-diversified such that the connector sequence of the nucleic acid molecule of the first plurality of nucleic acid molecules is different from other connector sequences of other nucleic acid molecules of the first plurality of nucleic acid molecules. In some embodiments, the nucleic acid molecule of the first plurality of nucleic acid molecules further comprises a first J region of the first TCR chain and/or a second J region of the second TCR chain. In some embodiments, (i) the first TCR chain is a TCR alpha chain and the second TCR chain is a TCR beta chain or (ii) the first TCR chain is a TCR gamma chain and the second TCR chain is a TCR delta chain. In some embodiments, the TCR V gene is a TRAV gene, a TRBV gene, a TRGV gene, or a TRDV gene. In some embodiments, the nucleic acid molecule of the second plurality of nucleic acid molecules is a double-stranded nucleic acid molecule. In some embodiments, the nucleic acid molecule of the second plurality of nucleic acid molecules further comprises a sequence encoding a portion of a self-cleaving peptide. In some embodiments, the anti-connector sequence is an overhang of the nucleic acid molecule of the second plurality of nucleic acid molecules. In some embodiments, the connector sequence or the anti-connector sequence is at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, 150, 200, or more nucleotides in length. In some embodiments, the method further comprises (i) extending a 3′ end of the nucleic acid molecule of the first plurality of nucleic acid molecules hybridized thereto with the nucleic acid molecule of the second plurality of nucleic acid molecules and/or (ii) extending a 3′ end of the nucleic acid molecule of the second plurality of nucleic acid molecules hybridized thereto with the nucleic acid molecule of the first plurality of nucleic acid molecules. In some embodiments, the method further comprises ligating the nucleic acid molecule of the first plurality of nucleic acid molecules with the nucleic acid molecule of the second plurality of nucleic acid molecule.

In some embodiments, the method further comprises contacting the nucleic acid molecule comprising the sequence encoding the first CDR3 and the second CDR3 and the sequence derived from the TCR V gene with a restriction enzyme to generate a sticky end. In some embodiments, the method further comprises contacting the nucleic acid molecule comprising the sequence encoding the first CDR3 and the second CDR3 and the sequence derived from the TCR V gene with an additional nucleic acid molecule comprising a sequence encoding a constant region or portion thereof. In some embodiments, the method further comprises ligating the nucleic acid molecule comprising the sequence encoding the first CDR3 and the second CDR3 and the sequence derived from the TCR V gene with the additional nucleic acid molecule through the sticky end. In some embodiments, the sequence encoding the first CDR3 and the second encoding the second CDR3 are separated by at most about 100, 90, 80, 70, 60, 50, 40, 30, 20, 10, or 5 nucleotides. In some embodiments, the sequence derived from the TCR V gene comprises a sequence encoding FR1, CDR1, FR2, CDR2, and FR3. In some embodiments, the sequence derived from the TCR V gene comprises a sequence encoding L-PART1, L-PART2, FR1, CDR1, FR2, CDR2, and FR3.

In another aspect, a method for generating a plurality of nucleic acid molecules, each nucleic acid molecule of the plurality encoding a T-cell receptor (TCR) chain or region thereof, can comprise: contacting a first plurality of nucleic acid molecules and a second plurality of nucleic acid molecules to generate a third plurality of nucleic acid molecules comprising at least two different nucleic acid molecules, wherein each of the at least two different nucleic acid molecules has a different sequence encoding a different TCR chain or region thereof, and wherein the at least two different nucleic acid molecules are generated in a same compartment.

In some embodiments, each nucleic acid molecule of the first plurality of nucleic acid molecules comprises a sequence encoding a CDR3 of the TCR chain. In some embodiments, each nucleic acid molecule of the first plurality of nucleic acid molecules comprises a J region of the TCR chain. In some embodiments, each nucleic acid molecule of the second plurality of nucleic acid molecules comprises a sequence derived from a TCR V gene of the TCR chain. In some embodiments, the TCR V gene is a human TCR V gene. In some embodiments, the TCR V gene is a human TRAV1-1, TRAV1-2, TRAV2, TRAV3, TRAV4, TRAV5, TRAV6, TRAV7, TRAV8-1, TRAV8-2, TRAV8-3, TRAV8-4, TRAV8-6, TRAV9-1, TRAV9-2, TRAV10, TRAV12-1, TRAV12-2, TRAV12-3, TRAV13-1, TRAV13-2, TRAV14, TRAV16, TRAV17, TRAV18, TRAV19, TRAV20, TRAV21, TRAV22, TRAV23, TRAV24, TRAV25, TRAV26-1, TRAV26-2, TRAV27, TRAV29, TRAV30, TRAV34, TRAV35, TRAV36, TRAV38-1, TRAV38-2, TRAV39, TRAV40, or TRAV41. In some embodiments, the TCR V gene is a human TRBV2, TRBV3-1, TRBV4-1, TRBV4-2, TRBV4-3, TRBV5-1, TRBV5-4, TRBV5-5, TRBV5-6, TRBV5-8, TRBV6-1, TRBV6-2, TRBV6-3, TRBV6-4, TRBV6-5, TRBV6-6, TRBV6-8, TRBV6-9, TRBV7-2, TRBV7-3, TRBV7-4, TRBV7-6, TRBV7-7, TRBV7-8, TRBV7-9, TRBV9, TRBV10-1, TRBV10-2, TRBV10-3, TRBV11-1, TRBV11-2, TRBV11-3, TRBV12-3, TRBV12-4, TRBV12-5, TRBV13, TRBV14, TRBV15, TRBV16, TRBV18, TRBV19, TRBV20-1, TRBV24-1, TRBV25-1, TRBV27, TRBV28, TRBV29-1, or TRBV30. In some embodiments, the sequence derived from the TCR V gene comprises a sequence encoding FR1, CDR1, FR2, CDR2, and FR3. In some embodiments, the sequence derived from the TCR V gene comprises a sequence encoding L-PART1, L-PART2, FR1, CDR1, FR2, CDR2, and FR3. In some embodiments, the TCR chain is a TCR alpha chain, a TCR beta chain, a TCR gamma chain, or a TCR delta chain. In some embodiments, each nucleic acid molecule of the first plurality of nucleic acid molecules further comprises an additional sequence encoding an additional CDR3 of an additional TCR chain. In some embodiments, each nucleic acid molecule of the first plurality of nucleic acid molecules comprises an additional J region of the additional TCR chain. In some embodiments, the TCR chain and the additional TCR chain are a cognate pair of TCR chains. In some embodiments, a nucleic acid molecule of the plurality of nucleic acid molecules encodes a different TCR or portion thereof. In some embodiments, a given nucleic acid molecule of the first plurality of nucleic acid molecules comprises a connector sequence, which connector sequence is usable for linking the given nucleic acid molecule of the first plurality of nucleic acid molecules to a given nucleic acid molecule of the second plurality of nucleic acid molecules. In some embodiments, the given nucleic acid molecule of the first plurality of nucleic acid molecules and the given nucleic acid molecule of the second plurality of nucleic acid molecules encodes a functional TCR chain or portion thereof. In some embodiments, the given nucleic acid molecule of the second plurality of nucleic acid molecules comprises an anti-connector sequence, which anti-connector sequence is complementary to the connector sequence of the given nucleic acid molecule of the first plurality of nucleic acid molecules. In some embodiments, the method further comprises linking the given nucleic acid molecule of the first plurality of nucleic acid molecules and the given nucleic acid molecule of the second plurality of nucleic acid molecules. In some embodiments, linking comprises hybridizing the given nucleic acid molecule of the first plurality of nucleic acid molecules and the given nucleic acid molecule of the second plurality of nucleic acid molecules. In some embodiments, hybridizing comprises hybridizing the connector sequence of the given nucleic acid molecule of the first plurality of nucleic acid molecules with the anti-connector sequence of the given nucleic acid molecule of the second plurality of nucleic acid molecules. In some embodiments, the method further comprises (i) extending a free 3′ end of the given nucleic acid molecule of the second plurality of nucleic acid molecules using the given nucleic acid molecule of the first plurality of nucleic acid molecules as a template, and/or (ii) extending a free 3′ end of the nucleic acid molecule of the first plurality of nucleic acid molecules using the given nucleic acid molecule of the second plurality of nucleic acid molecules as a template, to generate a nucleic acid molecule of the third plurality of nucleic acid molecules. In some embodiments, the method further comprises ligating the given nucleic acid molecule of the first plurality of nucleic acid molecules and the given nucleic acid molecule of the second plurality of nucleic acid molecules. In some embodiments, the method further comprises contacting the nucleic acid molecule of the third plurality of nucleic acid molecules with a restriction enzyme to generate a sticky end. In some embodiments, the method further comprises contacting the nucleic acid molecule of the third plurality of nucleic acid molecules with an additional nucleic acid molecule. In some embodiments, the additional nucleic acid molecule encodes a constant region or a portion thereof of a TCR chain. In some embodiments, the method further comprises ligating the nucleic acid molecule of the third plurality of nucleic acid molecules and the additional nucleic acid molecule. In some embodiments, at least five (e.g., in some cases, at least about 6, 7, 8, 9, 10, 20, 30, 40, 50, 60, 70, 80, 90, 100, 200, 300, 400, 500, 1,000, 2,000, 3,000, 4,000, 5,000, 10,000, 20,000, 30,000, 40,000, or more) different nucleic acid molecules of the third plurality of nucleic acid molecules are generated in the same compartment. In some embodiments, at least ten different nucleic acid molecules of the third plurality of nucleic acid molecules are generated in the same compartment. In some embodiments, the same compartment is a well, a tube, or a droplet.

In another aspect, a method for generating a plurality of nucleic acid molecules can comprise: (a) providing a first plurality of nucleic acid molecules, wherein a nucleic acid molecule of the first plurality of nucleic acid molecules comprises a sequence encoding a first CDR3 of a first T-cell receptor (TCR) chain and a second CDR3 of a second TCR chain, wherein the first CDR3 and the second CDR3 are from a cognate pair of TCR chains; (b) providing a second plurality of nucleic acid molecules, wherein a nucleic acid molecule of the second plurality of nucleic acid molecules comprises a sequence derived from a TCR V gene; and (c) contacting the first plurality of nucleic acid molecules and the second plurality of nucleic acid molecules, wherein the nucleic acid molecule of the first plurality of nucleic acid molecules links with the nucleic acid molecule of the second plurality of nucleic acid molecules to form a linear nucleic acid molecule comprising the sequence encoding the first CDR3 and the second CDR3 and the sequence derived from the TCR V gene, wherein the sequence encoding the first CDR3 and the second CDR3 and the TCR V gene are derived from the cognate pair of TCR chains.

In another aspect, a method for generating a plurality of nucleic acid molecules can comprise: (a) providing a first plurality of nucleic acid molecules, wherein a nucleic acid molecule of the first plurality of nucleic acid molecules comprises (i) a synthetic sequence encoding a first CDR3 of a first T-cell receptor (TCR) chain and a second CDR3 of a second TCR chain and (ii) a synthetic sequence encoding a third CDR3 of a third T-cell receptor (TCR) chain and a fourth CDR3 of a fourth TCR chain, wherein the first CDR3 and the second CDR3 are from a first cognate pair of TCR chains and wherein the third CDR3 and the fourth CDR3 are from a second cognate pair of TCR chains; (b) providing a second plurality of nucleic acid molecules, wherein a nucleic acid molecule of the second plurality of nucleic acid molecules comprises a sequence derived from a TCR V gene; and (c) contacting the first plurality of nucleic acid molecules and the second plurality of nucleic acid molecules, wherein the nucleic acid molecule of the first plurality of nucleic acid molecules links with the nucleic acid molecule of the second plurality of nucleic acid molecules to form a nucleic acid molecule comprising the sequence encoding the first CDR3 and the second CDR3 and the sequence derived from the TCR V gene, wherein the sequence encoding the first CDR3 and the second CDR3 and the TCR V gene are derived from the cognate pair of TCR chains.

In another aspect, a method for generating a nucleic acid molecule encoding a T-cell receptor (TCR) chain or portion thereof can comprise: (a) providing at least one nucleic acid molecule comprising a sequence encoding a CDR3 of a TCR chain; (b) providing a plurality of nucleic acid molecules, each nucleic acid molecule of the plurality comprising a sequence derived from a TCR V gene, wherein the plurality of nucleic acid molecules comprises at least two different sequences derived from at least two different TCR V genes; and (c) contacting the at least one nucleic acid molecule of (a) to the plurality of nucleic acid molecules of (b) in a same compartment, wherein the at least one nucleic acid molecule of (a) is capable of linking to a nucleic acid molecule of the plurality of nucleic acid molecules to generate a third nucleic acid molecule comprising the sequence encoding the CDR3 and a sequence derived from one of the at least two different TCR V genes, thereby generating the nucleic acid molecule encoding the TCR chain or portion thereof.

In some embodiments, the at least one nucleic acid molecule comprises a first plurality of nucleic acid molecules, wherein each nucleic acid molecule of the first plurality of nucleic acid molecules comprises a sequence encoding a CDR3 of a TCR chain. In some embodiments, the at least one nucleic acid molecule of (a) is capable of specifically linking to a nucleic acid molecule of the plurality of nucleic acid molecules that comprises a sequence derived from any single given TCR V gene of the at least two different TCR V genes. In some embodiments, the at least one nucleic acid molecule further comprises a J region of the TCR chain. In some embodiments, each nucleic acid molecule of the first plurality of nucleic acid molecules further comprises a J region of a TCR chain. In some embodiments, the at least two TCR V genes are human TCR V genes or mouse TCR V genes. In some embodiments, the at least two TCR V genes are selected from the group consisting of a human TRAV1-1, TRAV1-2, TRAV2, TRAV3, TRAV4, TRAV5, TRAV6, TRAV7, TRAV8-1, TRAV8-2, TRAV8-3, TRAV8-4, TRAV8-6, TRAV9-1, TRAV9-2, TRAV10, TRAV12-1, TRAV12-2, TRAV12-3, TRAV13-1, TRAV13-2, TRAV14, TRAV16, TRAV17, TRAV18, TRAV19, TRAV20, TRAV21, TRAV22, TRAV23, TRAV24, TRAV25, TRAV26-1, TRAV26-2, TRAV27, TRAV29, TRAV30, TRAV34, TRAV35, TRAV36, TRAV38-1, TRAV38-2, TRAV39, TRAV40, and TRAV41. In some embodiments, the at least two TCR V genes are selected from the group consisting of a human TRBV2, TRBV3-1, TRBV4-1, TRBV4-2, TRBV4-3, TRBV5-1, TRBV5-4, TRBV5-5, TRBV5-6, TRBV5-8, TRBV6-1, TRBV6-2, TRBV6-3, TRBV6-4, TRBV6-5, TRBV6-6, TRBV6-8, TRBV6-9, TRBV7-2, TRBV7-3, TRBV7-4, TRBV7-6, TRBV7-7, TRBV7-8, TRBV7-9, TRBV9, TRBV10-1, TRBV10-2, TRBV10-3, TRBV11-1, TRBV11-2, TRBV11-3, TRBV12-3, TRBV12-4, TRBV12-5, TRBV13, TRBV14, TRBV15, TRBV16, TRBV18, TRBV19, TRBV20-1, TRBV24-1, TRBV25-1, TRBV27, TRBV28, TRBV29-1, and TRBV30. In some embodiments, each sequence of the plurality of sequences derived from the at least two different TCR V genes comprises a sequence encoding L-PART1, L-PART2, FR1, CDR1, FR2, CDR2, and/or FR3. In some embodiments, the TCR chain is a TCR alpha chain, a TCR beta chain, a TCR gamma chain, or a TCR delta chain. In some embodiments, the at least one nucleic acid molecule further comprises an additional sequence encoding an additional CDR3 of an additional TCR chain. In some embodiments, the at least one nucleic acid molecule comprises an additional J region of the additional TCR chain. In some embodiments, the sequence encoding the CDR3 and the additional sequence encoding the additional CDR3 are separated by at most 100 nucleotides. In some embodiments, the TCR chain and the additional TCR chain are a cognate pair of TCR chains. In some embodiments, the at least one nucleic acid molecule comprises a connector sequence, which connector sequence is capable of linking the at least one nucleic acid molecule to the nucleic acid molecule of the plurality of nucleic acid molecules to generate the third nucleic acid molecule. In some embodiments, the at least one nucleic acid molecule and the nucleic acid molecule of the plurality of nucleic acid molecules encodes a functional TCR chain or portion thereof. In some embodiments, the nucleic acid molecule of the plurality of nucleic acid molecules comprises an anti-connector sequence, which anti-connector sequence is complementary to the connector sequence of the at least one nucleic acid molecule of (a). In some embodiments, the method further comprises linking the at least one nucleic acid molecule of (a) and the nucleic acid molecule of the plurality of nucleic acid molecules of (b). In some embodiments, linking comprises hybridizing the at least one nucleic acid molecule of (a) and the nucleic acid molecule of the plurality of nucleic acid molecules of (b). In some embodiments, hybridizing comprises hybridizing the connector sequence of the at least one nucleic acid molecule of (a) with the anti-connector sequence of the nucleic acid molecule of the plurality of nucleic acid molecules of (b). In some embodiments, the method further comprises (i) extending a free 3′ end of the nucleic acid molecule of the plurality of nucleic acid molecules using the at least one nucleic acid molecule of (a) as a template, and/or (ii) extending a free 3′ end of the at least one nucleic acid molecule of (a) using the nucleic acid molecule of the plurality of nucleic acid molecules as a template, to generate the third nucleic acid molecule. In some embodiments, the method further comprises ligating the at least one nucleic acid molecule of (a) and the nucleic acid molecule of the plurality of nucleic acid molecules (b). In some embodiments, the method further comprises contacting the third nucleic acid molecule with a restriction enzyme to generate a sticky end. In some embodiments, the method further comprises contacting the third nucleic acid molecule with an additional nucleic acid molecule. In some embodiments, the additional nucleic acid molecule encodes a constant region or portion thereof of a TCR chain. In some embodiments, the method further comprises ligating the third nucleic acid molecule and the additional nucleic acid molecule. In some embodiments, a plurality of nucleic acid molecules, each encoding a different TCR chain or portion thereof, are generated in the same compartment. In some embodiments, at least five different nucleic acid molecules of the plurality of nucleic acid molecules are generated in the same compartment. In some embodiments, at least ten different nucleic acid molecules of the plurality of nucleic acid molecules are generated in the same compartment. In some embodiments, the same compartment is a well, a tube, or a droplet.

Additional details on TCR gene assembly methods can be found in International Application PCT/US2020/026558, which is entirely incorporated herein by reference.

### Pharmaceutical Compositions

The present disclosure also provides pharmaceutical compositions comprising a TCR or a cell expressing a TCR identified by the methods described herein in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure can be formulated for intravenous administration.

Pharmaceutical compositions of the present disclosure may be administered in a manner appropriate to the disease to be treated (or prevented). The quantity and frequency of administration can be determined by such factors as the condition of the patient, and the type and severity of the patient's disease, although appropriate dosages may be determined by clinical trials.

The pharmaceutical composition can be substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some cases, the bacterium can be at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, Streptococcus pyogenes group A, and any combinations thereof.

### EXAMPLES

### Example 1: High-Throughput Identification of Neoantigen-Reactive TCRs from TILs

Tumor tissue can be surgically removed from a cancer patient. Within 24 hours after surgery, 0.5 to 3 cm³ of tumor tissue can be minced on ice. Single-cell suspension can be obtained using established enzymatic treatment (e.g., using collagenase and Dnase I) and optionally using tissue-dissociating instruments such as GentleMACS. Cell strainer can be used to move large debris. Single cells (e.g., live single cells) can be isolated using FACS. T cells (e.g., live T cell) can be isolated using a variety of positive-selection or negative-selection methods based on FACS or MACS. The T cells can be used as input for various sequencing methods to determine paired TCR chains within each T cell. The various sequencing methods include, but are not limited to, Sanger sequencing, high-throughput sequencing, sequencing-by-synthesis, single-molecule sequencing, sequencing-by-ligation, RNA-Seq, Next generation sequencing (NGS), Digital Gene Expression, Clonal Single MicroArray, shotgun sequencing, Maxim-Gilbert sequencing, or massively-parallel sequencing. The T cells can be used as input for single-cell RNA-Seq methods such as inDrop or DropSeq. Briefly, each cell will be encapsulated in a droplet or a microwell. A polynucleotide barcode unique to each cell (e.g., present on the reverse-transcription primer or template-switching oligonucleotide) will be introduced to many mRNA molecules from the same cell. The transcriptional profile as well as the TCR sequences of the alpha and beta chain of each cell can be obtained since they share the same polynucleotide barcode. The TCR alpha and beta chain sequences are therefore informatically linked. In a typical experiment, 1,000 to 5,000 informatically paired TCR sequences can be obtained. From each paired TCR sequence, the TRAV gene identity, CDR3-alpha sequence, TRAJ gene identity, TRBV gene identity, CDR3-beta sequence, and TRBJ gene identity can be extracted using publicly available software packages such as MiXCR. This information can be used to design a pool of paired CDR3J oligonucleotides (oligos) encoding all the 1,000 to 5,000 TCRs. These paired CDR3J oligos can be used to synthesize a pool of paired TCR-encoding polynucleotide using the TCR gene assembly methods described in U.S. Provisional Patent Applications No. 62/829,813, No. 62/838,465 and No. 62/898,053, and International Application PCT/US2020/026558, each of which is entirely incorporated herein by reference. This pool of paired TCR-encoding polynucleotides can be converted to a pool of lentiviral vectors using methods such as Gibson Assembly or Golden Gate assembly, where the full-length TCR beta chain and the full-length TCR alpha chain are linked via a P2A self-cleaving peptide, and the TCR is driven by a human EF1A promoter.

Alternatively, instead of informatically pairing the TCR alpha chain and beta chain and synthesizing paired TCRs in vitro, the TCR alpha chain and beta chain can be linked within an individual compartment for each single T cell. For example, each T cell of a plurality T cells can be sequestered within an individual compartment such as a droplet or a hydrogel particle, and the mRNA of each TCR chain can be reverse transcribed and amplified. The amplification products of the TCR alpha chain and beta chain can then be physically linked by methods such as ligation or overlapping PCRs. Methods for physically linking the TCR chains are described in U.S. Provisional Patent Application No. 62/718,227, filed Aug. 13, 2018, U.S. Provisional Patent Application No. 62/725,842, filed Aug. 31, 2018, U.S. Provisional Patent Application No. 62/732,898, filed Sep. 18, 2018, U.S. Provisional Patent Application No. 62/818,355, filed Mar. 14, 2019, and U.S. Provisional Patent Application No. 62/823,831, filed Mar. 26, 2019, and International Application PCT/US2019/046170, each of which is entirely incorporated herein by reference.

The lentiviral vector can be used to transduce a Jurkat 76-based reporter cell line (Rosskopf et al., Oncotarget 2017) where TCR signaling leads to expression of a fluorescent protein. The transduction can be performed at low multiplicity of infection (MOI) so that the majority of transduced reporter cell expresses only one TCR. The TCR-expressing reporter cells can be considered TCR-engineered reporter cells. These cells can also be regarded as pre-selection TCR-programmed recipient cells. The total DNA or RNA of an aliquot of these cells can be isolated and the exogenous TCR loci of these cells can be amplified and subject to NGS to measure the frequency or relative abundance of each TCR in the pre-selection TCR-programmed recipient cells.

Another part of the resected tumor tissue can be used to prepare genomic DNA and RNA for whole exome sequencing (WES) and whole transcriptome sequencing (RNA-Seq), respectively.

To generate the APC, two options can be considered. Autologous APC can be obtained by transforming autologous B cells into B-lymphoblastoid cells (B-LCLs), or by differentiating autologous monocytes into monocyte derived dendritic cells (MDDCs). Alternatively, artificial APCs (aAPCs) can be used. To do so, the genotypes of Class I MHC genes of the patient can be obtained from WES and transcriptome sequencing data. All genes encoding such MHC genes can be synthesized and mRNAs encoding these genes can be obtained with in vitro transcription, enzymatic capping and enzymatic polyadenylation. The resultant mRNAs can be electroporated into an aAPC cell line devoid of endogenous MHC expression (e.g., K562 cell line). The K562 cell line can be engineered to express co-stimulatory molecules (Butler and Hirano, Immunological Reviews 2014).

A pool of minigenes, tandem minigenes, or pool of tandem minigenes, where each minigene encodes a 25-mer mutated peptide discovered in the WES and RNA-Seq of the tumor sample, with the mutated residue at the 13th position, can be prepared in the form of plasmid (Pasetto et al., Cancer Immunol Res. 2016) or mRNA (Kreiter et al., J Immunol. 2008; Sahin et al., Nature 2017; Lu et al., Mol Ther. 2018), and transfected or electroporated into the APC. The APC can be co-cultured with the TCR-engineered reporter cells described above. After 12 to 24 hours, reporter cells that have upregulated level of fluorescent protein expression can be isolated by FACS. This selection can be regarded as the intended selection. These isolated cells can be regarded as the first plurality of post-selection TCR-programmed recipient cells.

Another pool of minigenes, tandem minigenes of pool of tandem minigenes, where each minigene encode a 25-mer wildtype counterpart of the mutated peptide described above, can be similarly prepared, and transfected or electroporated into the APC. The APC can be co-cultured with the TCR-engineered reporter cells described above. After 12 to 24 hours, reporter cells that have upregulated level of fluorescent protein expression can be isolated by FACS. This selection can be regarded as the negative-control selection. These isolated cells can be regarded as the second plurality of post-selection TCR-programmed recipient cells.

The total DNA or RNA of an aliquot of the first and the second plurality of post-selection TCR-programmed recipient cells can be isolated separately, and the exogenous TCR loci of these cells can be amplified and subject to NGS to measure the frequency or relative abundance of each TCR in the first and second pluralities of post-selection TCR-programmed recipient cells.

From the NGS data, TCRs whose frequency or relative abundance in the first pluralities of post-selection TCR-programmed recipient cells is significantly higher than that in the pre-selection TCR-programmed recipient cells and in the second plurality of post-selection TCR-programmed recipient cells can be identified using established statistical analyses. These TCRs may be regarded as putative neoantigen-reactive TCR. FIG. 3 shows a workflow of the TCR identification process. The TCR frequency analysis in FIG. 3 comprises comparing a first frequency of a TCR in the post-selection TCR-programmed recipient cells after contacting with a first antigen and a second frequency of a TCR in the post-selection TCR-programmed recipient cells after contacting with a second antigen.

### Example 2: High-Throughput Identification of Tumor-Reactive TCRs from TILs

The methods to isolate T cells (e.g., live T cells) from tumor samples, to informatically link the sequences of TCR alpha chain and TCR beta chain, to obtain the pool of paired TCR-encoding polynucleotide, to obtain pre-selection TCR-programmed recipient cells, to obtain the frequency or relative abundance of each TCR in the pre-selection TCR-programmed recipient cells can be the same as described in Example 1.

Autologous tumor spheroids can be used as antigen-presenting cells. From surgically removed tumor, tumor spheroids can be obtained using established methods (Sant et al., Drug Discov Today Technol. 2017). The pre-selection TCR-engineered reporter cells can be co-cultured with the tumor spheroid so that the ratio of TCR-engineered reporter cell and tumor cell is 1:1. Anti-CD28 antibodies can be added in the co-culture to provide signal 2. After 12 to 24 hours of co-culture, TCR-engineered reporter cells with elevated level of fluorescent protein can be isolated as descried in Example 1. The frequency of each TCR in the post-selection TCR-programmed recipient cells can also be obtained as described in Example 1. TCRs whose frequency or relative abundance in the post-selection TCR-programmed recipient cells is significantly higher than that in the pre-selection TCR-programmed recipient cells can be identified using established statistical analyses. These TCRs may be regarded as putative tumor-reactive TCRs. FIG. 1 and FIG. 2 show example workflows of the TCR identification process. The TCR frequency analysis in FIG. 2 comprises comparing a first frequency of a TCR in the pre-selection TCR-programmed recipient cells and a second frequency of a TCR in the post-selection TCR-programmed recipient cells.

### Example 3: Enrichment of Model TCRs from a Synthetic TCR Library

To evaluate the feasibility, sensitivity and/or specificity of the high-throughput screening assay described herein, a library of ˜1000 full-length, expressible TCRs were synthesized. Within this library, four TCRs are well-studied TCRs with known targets (also referred to as model TCRs), the rest are primarily TCRs from a tumor sample with unknown reactivity. The four model TCRs are:

(1) DMF4, which reacts to an HLA-A*02:01-restricted MART-1 epitope,
(2) DMF5, which reacts to the same HLA-A*02:01-restricted MART-1 epitope as DMF4, but has been reported to have higher affinity,
(3) C4, which reacts to an HLA-A*02:01-restricted WT-1 epitope, and
(4) C4-DLT, which reacts to the same HLA-A*02:01-restricted WT-1 epitope as C4, but has been reported to have higher affinity.

All of the synthesized TCRs had murine constant regions. This TCR library was cloned into a lentiviral vector and was driven by an EF-1alpha promoter. The backbone of the lentiviral vector also contained a YFP-expressing cassette driven by a different promoter. A lentiviral particle mixture was prepared using this vector library and used to infect peripheral T cells from a donor at a functional multiplicity of infection (MOI) of ˜0.1. In other words, the lentiviral particles were titrated so that after infection, about 10% of T cells were YFP-positive. This low MOI was used to reduce the occurrence of T cells that has more than one exogenous TCRs functionally integrated in its genome. The murine TCR constant domain on the cell surface was stained and the murinized TCRs were readily detectable in most YFP-positive cells even though the TCRs were transcribed from only one genomic locus. The YFP-positive, CD8+ cells were FACS-sorted and expanded briefly with anti-CD3/CD28 beads. These engineered T cells are referred to as polyclonal TCR-T cells, or pre-selection polyclonal TCR-T cells. In this example, the experimental data confirmed that less than 1% of the pre-selection polyclonal TCR-T cells were CD137-positive.

Next, two versions of antigen-presenting cells were prepared. In the first version, K562 cells were electroporated with an mRNA encoding HLA-A*02:01 and mRNAs encoding the MART-1 epitope and the WT-1 epitope described above. In the second version, K562 cells were electroporated with an mRNA encoding HLA-A*02:01 and an mRNA encoding an irrelevant antigen (or control antigen). Next, two co-cultures were set up. The first co-culture, called the ‘real’ co-culture, comprised the polyclonal TCR-T cells and the first version of antigen-presenting cells. For example, the ‘real’ co-culture can comprise a target antigen (e.g., a TSA or TAA) against which a TCR is to be identified. The second co-culture, called the ‘mock’ co-culture, comprised the polyclonal TCR-T cells and the second version of antigen-presenting cells. After one day of co-culture, the CD137-positive T cells were sorted from other cells. These cells sorted from the ‘real’ and ‘mock’ co-cultures are termed post-real-selection polyclonal TCR-T cells and post-mock-selection polyclonal TCR-T cells, respectively.

The frequency of each TCR in the pre-selection, post-real-selection and post-mock-selection polyclonal TCR-T cells were examined by NGS. To do this, total RNA from the sorted cells were isolated, subject to reverse transcription (RT) using a gene-specific RT primer targeting the murine TRBC domain. Next, a second-strand synthesis (SSS) primer containing a unique molecular identifiers (UMI) was added. The SSS primer has a 5′ domain, an 8-nt random sequence serving as the UMI, and a 3′ gene-specific domain targeting the 5′UTR of the synthetic TCRs. One thermocycle of denaturation, annealing, and extension was carried out in the presence of a thermophilic DNA polymerase (e.g., Q5) to complete the second strand synthesis. Next, a pair of primers targeting the 5′ domain of the SSS and a nested region (i.e., nested from the RT primer) on the murine TRBC domain was used to amplify the synthetic TCR beta chain cDNA. These cDNAs were sequences on Illumina NGS system (e.g., MiSeq, MiniSeq, HiSeq or NextSeq) to obtain the CDR3beta sequence on each cDNA molecule. The reads sharing the same UMI and same CDR3beta were digitally collapsed and counted as one original cDNA molecule. The original cDNA molecules with less than 10 reads were omitted. The frequency of a TCR can be defined as the number of original cDNA molecules encoding this TCR divided by the number of all original cDNA molecules observed in one sample.

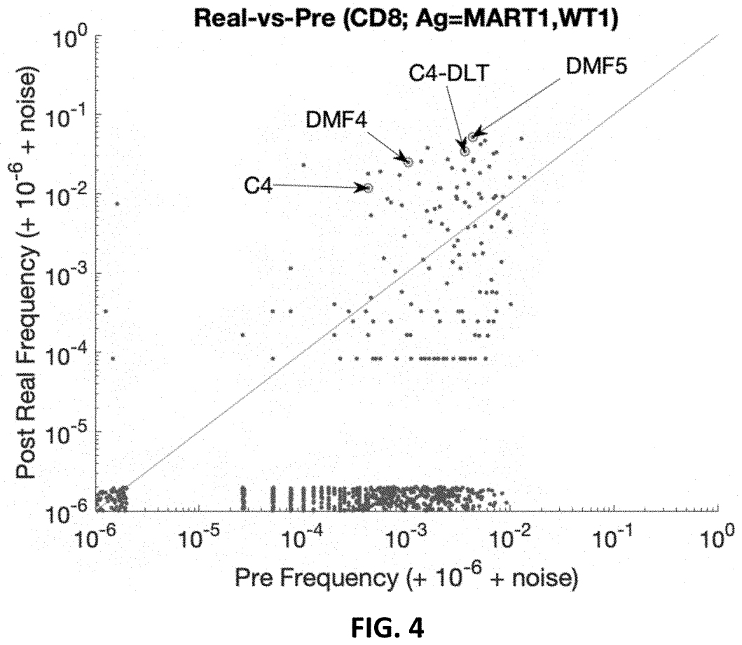

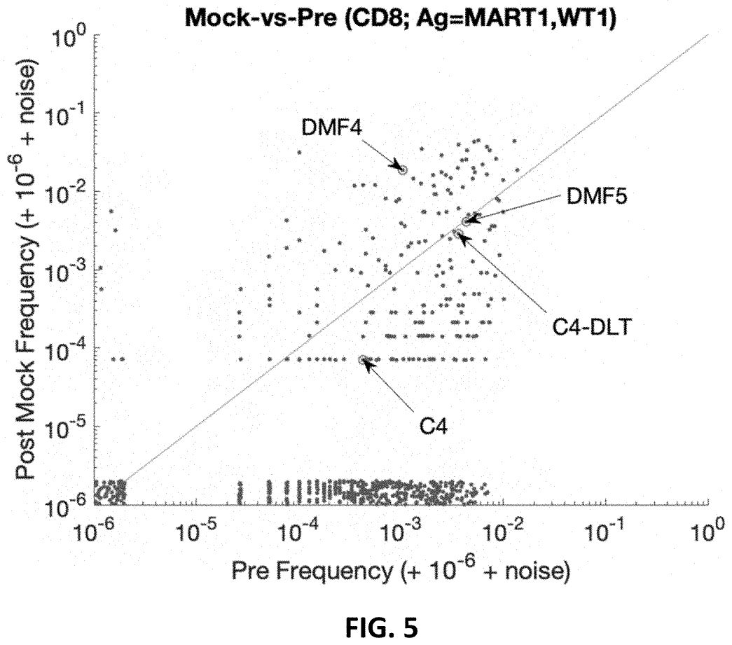

FIG. 4 shows the frequency of each TCR in the post-real-selection polyclonal TCR-T cells and pre-selection polyclonal TCR-T cells, which shows the enrichment of each TCR after the ‘real’ co-culture and sorting. A small number (1e⁻⁶) was added to each frequency value so that the dots that represent a frequency of zero can be shown on this plot in log scale. A random number in the range of 0 and 1e⁻⁶ was added to each frequency value so that the dots representing different TCRs that have the same frequency values in the X- and Y-axis are not completely overlapping. It can be seen that all four model TCRs showed ˜10-fold higher frequency in the post-real-selection polyclonal TCR-T cells than in the pre-selection polyclonal TCR-T cells. In contrast, most other TCRs were either undetectable in the post-real-selection polyclonal TCR-Ts or had a frequency that was less than 10× the frequency in the pre-selection polyclonal TCR-T cells (e.g., shows less than 10× enrichment). In contrast, after the ‘mock’ co-culture, these model TCRs, other than DMF4, did not show enrichment (FIG. 5). FIG. 5 shows the frequency of each TCR in the post-mock-selection polyclonal TCR-T cells (Y-axis) and pre-selection polyclonal TCR-T cells (X-axis). A small number (1e⁻⁶) and noise were added to each frequency as in FIG. 4. It is worth noting that DMF4 has shown considerable non-specific binding in our previous experiments, in that, T cells engineered with DMF4 shows upregulation of CD137 after co-culture with K562 cells transfected with HLA-A*02:01 mRNAs without the MART1 antigen. Therefore, the unwanted enrichment of DMF4 in the ‘mock’ co-culture and sorting was observed in this example.

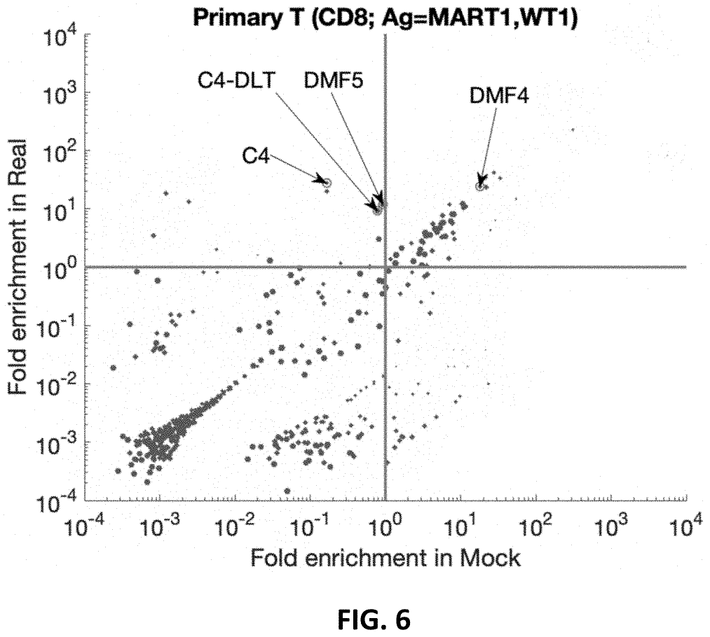

FIG. 6 shows the enrichment factor of TCR in the ‘real’ co-culture and the ‘mock’ co-culture. The enrichment factor in the ‘real’ co-culture can be defined as the frequency of a TCR in post-real-selection polyclonal TCR-T cells divided by that in the pre-selection polyclonal TCR-T cells. The enrichment factor in the ‘mock’ co-culture can be defined as the frequency of a TCR in post-mock-selection polyclonal TCR-T cells divided by that in the pre-selection polyclonal TCR-T cells. A small number (1e⁻⁶) and a random number between 0 and 1e⁻⁶ was added to all frequency values to facilitate visualization. Consistent with FIG. 4 and FIG. 5, the model TCRs C4, C4-DLT and DMF5 showed enrichment in the ‘real’ co-culture, while DMF4 showed enrichment in both the ‘real’ and the ‘mock’ co-cultures. These data demonstrate that functional, antigen-specific TCRs can be identified using the frequency-based screening methods described herein.

While various embodiments of the invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1.-54. (canceled)

55. A method for identifying a T-cell receptor (TCR), comprising:
(a) providing a plurality of T cells expressing a plurality of TCRs, wherein each T cell of the plurality of T cells expresses a cognate pair of a TCR of the plurality of TCRs;
(b) pairing a first polynucleotide encoding a first TCR chain of the cognate pair of the TCR of a given T cell of the plurality of T cells of (a) and a second polynucleotide encoding a second TCR chain of the cognate pair of the TCR of the given T cell of the plurality of T cells of (a), thereby generating a plurality of polynucleotide pairs;
(c) delivering polynucleotides comprising the sequences of the plurality of polynucleotide pairs generated in (b) into a plurality of recipient cells, wherein each recipient cell comprises a polynucleotide comprising the sequence of at least one polynucleotide pair of the plurality of polynucleotide pairs;
(d) expressing in the plurality of recipient cells the sequences of the plurality of polynucleotide pairs delivered into the plurality of recipient cells of (c);
(e) determining a TCR repertoire of the plurality of recipient cells of (d) by sequencing and determining a frequency of a TCR in the TCR repertoire in the plurality of recipient cells of (d);
(f) contacting the plurality of recipient cells of (d) with one or more antigens, thereby activating a marker in a subset of the plurality of recipient cells;
(g) isolating the subset of the plurality of recipient cells of (f) based on the marker;
(h) determining a TCR repertoire of the subset of the plurality of recipient cells of (g) by sequencing and determining a frequency of a TCR in the TCR repertoire in the subset of the plurality of recipient cells of (g); and
(i) identifying a TCR having a frequency in the subset of the plurality of recipient cells of (g) that is higher than its frequency in the plurality of recipient cells of (d).

56. The method of claim 55, wherein the frequency of the TCR in the subset of the plurality of recipient cells of (g) is at least 1.5-fold or more higher than its frequency in the plurality of recipient cells of (d).

57. The method of claim 55, wherein the marker is a T-cell activation marker.

58. The method of claim 55, wherein the marker is a reporter protein.

59. The method of claim 58, wherein the reporter protein is a fluorescent protein.

60. The method of claim 55, wherein the marker is a cell surface protein, an intracellular protein or a secreted protein.

61. The method of claim 60, wherein the marker is the intracellular protein or the secreted protein, and wherein the method further comprises, prior to isolating, fixing and/or permeabilizing the plurality of recipient cells.

62. The method of claim 60, wherein the secreted protein is a cytokine and wherein the cytokine is IFN-γ, TNF-alpha, IL-17A, IL-2, IL-3, IL-4, GM-CSF, IL-10, IL-13, granzyme B, perforin, or a combination thereof.

63. The method of claim 60, wherein the cell surface protein is CD39, CD69, CD103, CD25, PD-1, TIM-3, OX-40, 4-1BB, CD137, CD3, CD28, CD4, CD8, CD45RA, CD45RO, GITR, FoxP3, or a combination thereof.

64. The method of claim 55, wherein the one or more antigens are presented on one or more antigen presenting cells (APCs).

65. The method of claim 64, wherein (i) the one or more APCs are from one or more cells isolated from a subject or (ii) the one or more APCs are one or more artificial APCs (aAPCs).

66. The method of claim 64, wherein the one or more APCs express MHC molecules exogenous to the one or more APCs.

67. The method of claim 64, wherein the one or more APCs are one or more cancer cells, a tumorsphere, a tumoroid, or a derivative thereof.

68. The method of claim 64, wherein the one or more APCs comprise an antigen coding DNA or RNA.

69. The method of claim 64, wherein the one or more APCs are pulsed with the one or more antigens.

70. The method of claim 55, wherein the one or more antigens are one or more tumor antigens.

71. The method of claim 55, further comprising selecting the TCR identified in (i) from the TCR repertoire of the plurality of recipient cells of (d) or the TCR repertoire of the subset of the plurality of recipient cells of (g).

72. The method of claim 71, wherein selecting the TCR identified in (i) comprises amplifying the TCR.

73. The method of claim 55, wherein the polynucleotide in (c) comprises a barcode.

74. A pharmaceutical composition comprising a TCR or a cell expressing a TCR identified by the method of claim 55.
